(19) United States
(12) Patent Application Publication
(10) Pub. No.: US 2008/0015265 A1
(43) Pub. Date: Jan. 17, 2008

(54) Methods of treating obesity using satiety factors

(76) Inventors: Byron Rubin, Honeoye Falls, NY (US); Peter C.M. McWilliams, San Francisco, CA (US)

Correspondence Address: JONES DAY, 222 EAST 41ST ST, NEW YORK, NY, 10017, US

(21) Appl. No.: 11/827,275

(22) Filed: Jul. 10, 2007

Related U.S. Application Data
(60) Provisional application No. 60/830,410, filed on Jul. 11, 2006.
(60) Provisional application No. 60/899,223, filed on Feb. 2, 2007.

(51) Int. Cl. A61K 31/00; A61P 43/00; G01N 33/00

(52) U.S. Cl. 514/789; 435/4; 436/63

(57) ABSTRACT

The present invention provides methods of treating or preventing disorders or conditions associated with an undesirable level of a satiety factor by administering to a subject in need thereof an effective amount of an agonist or antagonist of a satiety factor. The present invention also provides methods of selecting a subject for therapy with an agonist or antagonist of a satiety factor. Exemplary disorders or conditions associated with an undesirable level of a satiety factor include overweight, obesity, metabolic disorders, hypertension, lipid related disorders, anorexia and type II diabetes.

### 1. CROSS REFERENCE TO RELATED APPLICATION

This application claims priority under 35 U.S.C. § 119(e) to U.S. Provisional Application Nos. 60/830,410, filed Jul. 11, 2006 and 60/899,223, filed Feb. 2, 2007.

### 2. FIELD OF THE INVENTION

The present invention provides methods of treating or preventing disorders or conditions associated with an undesirable level of a satiety factor, for example, a gut peptide, by administering to a subject in need thereof an effective amount of an agonist or antagonist of the satiety factor. The present invention also provides methods of selecting a subject for therapy with an agonist or antagonist of a satiety factor. The present invention further provides methods of treating a patient population, for instance, patients having an undesirable level of a satiety factor by administering to patients an effective amount of an agonist or antagonist of the satiety factor. Exemplary disorders or conditions associated with an undesirable level of a satiety factor include overweight, obesity, metabolic disorders, hypertension, lipid related disorders, anorexia and type II diabetes.

### 3. BACKGROUND OF THE INVENTION

Obesity is a complex condition that is increasingly affecting the population worldwide. According to the World Health Organization, in 1995 there were an estimated 200 million obese adults worldwide and another 18 million under-five children classified as overweight. As of 2000, the number of obese adults had increased to over 300 million. See Formiguera et al., 2004, Best Practice & Research Clinical Gastroenterology, 18:6, 1125-1146.

Overweight or obesity has been shown to increase risk for several diseases and health conditions, including hypertension, dyslipidemia (high total cholesterol or high levels of triglycerides), type II diabetes, coronary heart disease, stroke, gallbladder disease, osteoarthritis, sleep apnea and respiratory problems and some cancers (for example, endometrial, breast and colon). See, e.g., U.S. National Center for Chronic Disease Prevention and Health Promotion. Its health consequences range from increased risk of premature death to serious chronic conditions that reduce the overall quality of life.

Various therapies have been proposed or tested for the modulation of physiological processes that might lead to conditions such as overweight or obesity. See Orzano et al., 2004, J. Am. Board Fam. Pract. 17(5):359-69. Numerous satiety factors, such as, for example, gut peptides, which are synthesized and secreted by the gastrointestinal tract of humans, have been shown to regulate food intake and satiety signals. Thus, certain endogenous gut peptides have been studied as potential candidates for obesity treatment. Exemplary gut peptides include for example, ghrelin, cholecystokinin (CCK), pancreatic peptide (PP), peptide-YY (PYY), glucagon-like peptide 1 (GLP-1), etc.

However, investigations, including clinical trials, of most satiety factors have yielded ambiguous and often contradictory results. Thus, there is a need in the art for effective methods of treating obesity and related diseases. The present invention addresses such needs and provides methods of treating such diseases.

### 4. SUMMARY OF THE INVENTION

The present invention is based, in part, on the discovery that by evaluating the level of a satiety factor, for example, a gut peptide, it is possible to identify a population of subjects that is, or will be, statistically more responsive to satiety factor treatment than other populations.

In one aspect, the present invention provides methods of treating or preventing a disorder or condition in a subject with an undesirable level of a satiety factor. The undesirable level can be too low, or too high, depending on the subject, the peptide and the disorder or condition. The methods comprise the step of administering to the subject an agonist or antagonist of the satiety factor in an amount effective for treating or preventing the disorder or condition. The agonist or antagonist is administered in a pharmaceutically acceptable formulation by a pharmaceutically acceptable route of administration according to one of skill in the art.

While not intending to be bound by any particular theory of operation, it is believed that the most effective approach to treat conditions such as overweight or obesity is to apply specific therapies to specific patient populations. Advantageously, in certain embodiments, the present invention provides methods of selecting a population, for example a sub-population, of subjects suitable for treatment with an effective amount of a satiety factor agonist or antagonist according to a method described herein. In certain embodiments, the methods of the invention enhance, or even enable, the therapeutic or prophylactic activity of the satiety factor agonist or antagonist. In certain embodiments, the methods of the invention can reduce or avoid potential side effects of the satiety factor agonist or antagonist by identifying subjects that are not to be administered the satiety factor agonist or antagonist.

Exemplary disorders or conditions associated with an undesirable level of a satiety factor include, but are not limited to, overweight, obesity, metabolic disorders, hypertension, lipid related disorders, anorexia and type II diabetes.

In another aspect, the present invention provides methods of treating an undesirable level of a satiety factor in a subject in need thereof. The methods comprise the step of administering to the subject an agonist or antagonist of said satiety factor in an amount effective for the treatment.

In another aspect, the present invention provides methods of selecting a subject for therapy with an agonist or antagonist of a satiety factor. In certain embodiments, the methods comprise the step of determining the amount of a satiety factor in a sample from the subject. The subject is selected for treatment when the amount of said satiety factor in the sample of the subject is less than a normal value. Normal satiety factor values are described in detail below.

In further aspect, the present invention provides for methods of treating or preventing a disorder or condition associated with an undesirable level of a satiety factor. The methods comprise the step of selecting a subject with an undesirable level of a satiety factor for treatment and administering to the subject an agonist or antagonist of said satiety factor in an amount effective for treating or preventing the disorder or condition. The methods of selecting a subject with an undesirable level of a satiety factor and administering an agonist or antagonist of a satiety factor are described herein. In certain embodiments, one agonist or antagonist of said satiety factor is administered. In certain embodiments, a plurality of agonists or antagonists of said satiety factor is administered. In certain embodiments, a plurality of agonists or antagonists of one or more satiety factors is administered. In certain embodiments, the agonist or antagonist of a satiety factor is administered in combination with a second agent useful for treating or preventing the disorder or condition.

The satiety factor of the present invention can be any molecule that is, or should be endogenously produced by a subject and is capable of regulating appetite, food intake, energy intake or expenditure or satiety signal. The satiety factor of the present invention includes peptide and non-peptide satiety factor The peptide satiety factor can be a gut peptide, i.e., a peptide that is, or should be endogenously produced by the gastrointestinal tract, such as, for example, stomach, pancreas, intestine or colon, and is capable of regulating appetite, food intake, energy intake or expenditure or satiety signal. In some embodiments, the gut peptide can also be produced by another organ, for example the brain. In some embodiments, the gut peptide is selected from the group consisting of amylin, bombesin or bombesin-like peptide, cholecystokinin, enterostatin, ghrelin, insulin, proglucagon-derived peptides such as glucagon, glucagon-like peptide 1 or oxyntomodulin, obestatin, pancreatic polypeptide and peptide YY. In some embodiments, the gut peptide is enterostatin. In some embodiments, the gut peptide is other than enterostatin.

The peptide satiety factor can be a non-gut peptide, i.e., a peptide that is, or should be endogenously produced by an organ or tissue other than the gastrointestinal tract, such as, for example, the brain, liver, or adipose tissue, and is capable of regulating appetite, food intake, energy intake or expenditure or satiety signal.

Satiety factors of the present invention include but are not limited to adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β-endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), melanocyte stimulating hormone (MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptides including, for example, glucagon, glucagon-like peptide 1 (GLP-1), glucagon-like peptide 2 (GLP-2) and oxyntomodulin, glicentin, glicentin-related pancreatic peptide and major proglucagon fragment, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

The agonist of a satiety factor can be any agent that mimics the biological activities of the satiety factor, induces similar physiological effects of the satiety factor, enhances the duration of effects of the satiety factor, enhances a biological activity of the satiety factor or enhances the selectivity of the satiety factor. In some embodiments, an agonist of a satiety factor is the satiety factor itself. In other embodiments, an agonist of a satiety factor is an active fragment, analogue or derivative of the satiety factor.

The antagonist of a satiety factor can be any agent that inhibits the biological activities or physiological effect of a satiety factor. In some embodiments, an antagonist of a satiety factor is an antibody of the satiety factor. In some embodiments, an antagonist of a satiety factor is an inhibitor of the satiety factor. In some embodiments, an antagonist of a satiety factor is an inhibitor of the receptor of the satiety factor

The agonist or antagonist of said satiety factor can be administered by any route known to those of skill in the art, including but not limited to oral, intranasal, intrapulmonary, intravenous, subcutaneous, transdermal, intragastric, intraperitoneal, intracerebroventricular or rectal administration.

Whether a subject has an undesirable level of a satiety factor can be determined by any method available to those of skill in the art. Exemplary methods are described herein. In certain embodiments, a subject has an undesirable level of a satiety factor when the subject expresses or secretes a lower amount of a satiety factor than a control subject does. In certain embodiments, a subject has an undesirable level of a satiety factor when the subject expresses or secretes a higher amount of a satiety factor than a control subject does. Agonists of satiety factors are useful when the subject expresses or secretes a lower amount of a satiety factor than a control subject does. Antagonists of satiety factors are useful when the subject expresses or secretes a higher amount of a satiety factor than a control subject does.

Advantageously, the normal satiety factor value need not be determined by one carrying out a method of the invention. Instead, the normal satiety factor value can be identified by consulting knowledge or data available to those of skill in the art. Such data can be obtained from any source available to those of skill in the art including, for example, medical records, clinical trial data and the like. In certain embodiments, sources can be developed with the amounts of satiety factor collected by those of skill in the art according to methods described herein.

In certain embodiments, the normal satiety factor amount is from a control subject presenting no symptom of a disorder or condition associated with an undesirable level of one or more satiety factors. In some embodiments, the control subject is a healthy subject with normal weight. In some embodiments, the control subject is a lean individual of normal weight.

The amount of satiety factor in the subject can be determined according to any technique known to those of skill in the art without limitation. In certain embodiments, the technique for measuring satiety factor is not critical for the invention and need not even be carried out by one practicing methods herein. In certain embodiments, the amount of satiety factor in the sample of the subject is determined by a technique described herein followed by comparing the amount to a normal satiety factor value in order to determine whether to select the subject for treatment with an agonist or antagonist of a satiety factor. In certain embodiments, the amount of satiety factor in the sample of the subject is determined by spectrometry, chromatography, immunoassay or electrophoresis as described in detail below. In some preferred embodiments, the amount of satiety factor is determined by immunoassay. In one preferred embodiment, the immunoassay is ELISA.

The amount of satiety factor can be measured in any sample of the subject as provided herein. The sample can be a fluid or tissue sample as described herein. Processes for preparing the fluid or tissue, for example, processes for extracting or purifying satiety factor are described herein. The amount of the satiety factor can be determined at a time deemed useful by a practitioner of skill in the art. In certain embodiments, the amount is measured after food intake. In certain embodiments, the amount is measured before food intake. In certain embodiments, a ratio of the amounts when the subject is fasted to when the subject is fed. Detailed methods of measuring are provided below.

In certain embodiments, the amounts of a plurality of satiety factors of the subject can be measured. Where the subject has an undesirable amount of one of the satiety factors, the subject can be administered an agonist or antagonist of the satiety factor according to the methods described herein. Where the subject has an undesirable amount of a plurality of satiety factors, the subject can be administered a plurality of agonists or antagonists of the satiety factors according to the methods described herein. In advantageous embodiments, the amounts of a panel of satiety factors of the subject can be measured. From these amounts, a combination of agonists or antagonists of selected satiety factors can be administered to the subject according to the methods described herein. In particular embodiments, a personalized cocktail of agonists and antagonists can be selected for the subject based on the amounts of the panel of satiety factors.

In another aspect, the present invention provides for kits for selecting a subject for treatment of obesity with an agonist or antagonist of a satiety factor. In some embodiments, the kits comprise a device capable of containing a fluid of the subject and a reagent capable of detecting satiety factor in the fluid. In certain embodiments, the kits comprise a reagent capable of detecting a satiety factor and an effective amount of an agonist or antagonist of the satiety factor. The kits can further comprise a label or labeling with instructions for using the kits. In certain embodiments, the kits comprise a label or labeling with a normal satiety factor value.

### 5. DETAILED DESCRIPTION OF THE INVENTION

5.1. Definitions

As used herein, the following terms shall have the following meanings:

The term “subject” refers to animals such as mammals, including, but not limited to, primate (e.g., human), cow, sheep, goat, horse, dog, cat, rabbit, rat, mouse and the like. In preferred embodiments, the subject is a human.

The term “satiety factor” refers to any molecule that is, or should be endogenously produced by a subject and is capable of regulating appetite, food intake, energy intake or expenditure or satiety signal. The satiety factor of the present invention can be peptide or non-peptide satiety factor. The peptide satiety factor of the present invention can be a gut peptide or a non-gut peptide as described in detail below.

Satiety factors of the present invention include but are not limited to adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β-endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), melanocyte stimulating hormone (MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

The term “gut peptide” refers to any peptide known in the art that is, or should be, produced endogenously by the gastrointestinal tract, such as, for example, stomach, pancreas, intestine or colon, and capable of regulating appetite, food intake, energy intake, energy expenditure, or satiety. In certain embodiments, a gut peptide can also be produced in another organ such as, for example, the brain. The gut peptides of the present invention include but are not limited to amylin, bombesin or bombesin-like peptide, cholecystokinin, enterostatin, ghrelin, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, obestatin, oxyntomodulin, pancreatic polypeptide and peptide YY.

In some embodiments, the gut peptide is selected from the group consisting of amylin, bombesin or bombesin-like peptide, cholecystokinin, enterostatin, ghrelin, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, obestatin, oxyntomodulin, pancreatic polypeptide and peptide YY. In some embodiments, the gut peptide is enterostatin. In some embodiments, the gut peptide is other than enterostatin.

In some embodiments, the gut peptide is selected from the group consisting of, amylin, bombesin or bombesin-like peptide, cholecystokinin, ghrelin, glucagon-like peptide 1, obestatin, oxyntomodulin, pancreatic polypeptide and peptide YY. In some embodiments, the gut peptide is amylin. In some embodiments, the gut peptide is bombesin or bombesin-like peptide. In some embodiments, the gut peptide is cholecystokinin. In some embodiments, the gut peptide is ghrelin. In some embodiments, the gut peptide is proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment. In some embodiments, the gut peptide is GLP-1. In some embodiments, the gut peptide is oxyntomodulin. In some embodiments, the gut peptide is obestatin. In some embodiments, the gut peptide is peptide YY. In some embodiments, the gut peptide is pancreatic peptide.

The term “non-gut peptide” refers to any peptide known in the art that is, or should be, produced endogenously by an organ or tissue other than the gastrointestinal tract, such as, for example, the brain, liver or adipose tissue, and capable of regulating appetite, food intake, energy intake, energy expenditure, or satiety.

In some embodiments, the non-gut peptide is selected from the group consisting of adiponectin, agouti-related protein (AGRP), apolipoprotein A-IV, beacon, brain derived neural factor (BDNF), calcitonin-gene related peptide (CGRP), β casomorphin, ciliary neurotrophic factor (CNTF), cocaine and amphetamine regulated transcript (CART), corticotropin-releasing hormone (CRH), cyclo-his-pro, dynorphin, β-endorphin, galanin, galanin-like peptide (GALP), growth hormone-releasing hormone (GHRH), hypocretins/orexins, insulin, insulin like growth factor I and II (IGF-I and IGF-II), leptin, melanin concentrating hormone (MCH), melanocyte stimulating hormone (MSH), motilin, nesfatin, neuromedin B and neuromedin U, neuropeptide B (NPB) and (NPW), neuropeptide K (NPK), neuropeptide Y (NPY), neurotensin (NT), oxytocin, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, glicentin, glicentin-related pancreatic peptide, major proglucagon fragment, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

The term “an agonist of a satiety factor” can be any agent that mimics the biological activity of, induces similar physiological effect of, or enhances the duration of effects, biological activity or selectivity of a satiety factor. In some embodiments, an agonist of a satiety factor is the satiety factor itself. In other embodiments, an agonist of a satiety factor is an active fragment, analogue or derivative of the satiety factor. An agonist of a satiety factor can also act through indirect action. For example, an agonist of a glucagon-like peptide 1 (GLP-1), can be an inhibitor of the enzyme dipeptidyl pepdiase IV (DPP IV), which inactivates GLP-1.

The term “an antagonist of a satiety factor” can be any agent that inhibits the biological activity or physiological effect of a satiety factor. In some embodiments, an antagonist of a satiety factor completely inhibits the biological activity or physiological effect of a satiety factor. In other embodiments, an antagonist of a satiety factor partially inhibits the biological activity or physiological effect of a satiety factor.

The term “undesirable level of satiety factor” refers to a subject that expresses or secretes a lower or higher amount of satiety factor than expected for the subject according to the judgment of one of skill in the art. In certain embodiments, a subject has an undesirable level of a satiety factor when the subject expresses or secretes a lower or higher amount of satiety factor in a fasting state than a control subject does. In certain embodiments, a subject has an undesirable level of a satiety factor when expresses or secretes a lower or higher amount of satiety factor after a meal than a control subject does.

In certain embodiments, a subject has an “undesirable level of a satiety factor” when the subject expresses or secretes a lower amount of a satiety factor than expected for the subject according to the judgment of one of skill in the art. In some embodiments, a subject has an undesirable level of a satiety factor when the subject does not express or secrete an amount of the satiety factor that is detectable using techniques available in the art. In some embodiments, a subject has an undesirable level of a satiety factor when the subject does not express or secrete any said satiety factor.

In certain embodiments, subject has an “undesirable level of a satiety factor” when the subject expresses or secretes a higher amount of a satiety factor than expected for the subject according to the judgment of one of skill in the art.

Whether a subject has an undesirable level of a satiety factor can be determined by techniques known to those of skill in the art. In certain embodiments, it is determined by measuring the amount of satiety factor in a sample from the subject and comparing such with a normal satiety factor value. In some embodiments, the subject has an undesirable level of a satiety factor when the amount of the satiety factor in the sample from the subject is less than 95%, 90%, 85%, 80%, 75%, 70%, 65%, 60%, 55%, 50%, 45%, 40%, 35%, 30%, 25%, 20%, 15%, 10%, 5%, 2% or 1% of a normal satiety factor amount according to the judgment of a practitioner of skill in the art. In other embodiments, the subject has an undesirable level of a satiety factor when the amount of the satiety factor in the sample from the subject is about 110%, 120%, 130%, 140%, 150%, 160%, 170%, 180%, 190%, 200%, 250%, 300%, 400%, 500%, 600%, 700%, 800%, 900%, 1000%, or 1500% of a normal satiety factor amount according to the judgment of a practitioner of skill in the art. Normal satiety factor amounts are described in the sections below.

The term a “control subject” is a subject that presents no symptoms of one or more disorders or conditions associated with an undesirable level of a satiety factor according to standards recognized by those of skill in the art. In some embodiments, a control subject has age, height, race and gender similar to a subject to be selected for treatment with an agonist or antagonist of a satiety factor. In some embodiments, the control subject is a lean subject or a subject with normal weight. When the subject is human, the control subject can be an individual with a Body Mass Index (“BMI”) range of 20-25 kg/m². BMI can be obtained by dividing body weight (in kilograms) by height (in meters) squared. A control subject is useful for establishing a normal satiety factor value that can be used to evaluate whether a subject has an undesirable level of a satiety factor.

“Preventing” or “prevention” refers to a reduction in the risk of acquiring a disease or disorder (i.e., causing at least one of the clinical symptoms of the disease not to develop in a subject that may be exposed to or predisposed to the disease but does not yet experience or display symptoms of the disease). Preferably, prevention refers to the use of a compound or composition in a subject not yet affected by the disease or disorder or not yet exhibiting a symptom of the disease or disorder, for instance a subject not yet infected or not yet exhibiting the symptoms of infection.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

The term “an effective amount” means an amount of an agonist or antagonist satiety factor or compositions comprising thereof that when, administered to a subject for treating a disease is sufficient to effect such treatment for the disease. An effective amount can vary depending on, inter alia, the satiety factor used, the disease and its severity and the age, weight, etc. of the subject to be treated.

The term “obesity” refers to a subject having weight and body mass particularly of fat tissue above currently accepted standards. In some embodiments, the subject is obese with a BMI above currently accepted standard. When a subject is a human, the current standards for both men and women accepted as “normal” are a BMI of 20-24.9 kg/m². In such embodiments, an obese subject has a BMI of 30 kg/m² or greater. In some embodiments, an obese subject has a BMI of 40 kg/m² or greater. In other embodiments, the subject is obese when it weighs more than 120% of the normal body weight for its age and height. Normal body weights vary among species and individuals based on height, body build, bone structure and sex.

The term “overweight” refers to a moderate excess of fat in a subject. In some embodiments, when a subject is a human, the overweight subject has a BMI of 25 kg/m² or greater.

The amino acid notations used herein for the twenty genetically encoded L-amino acids are conventional. In preferred embodiments, any peptide or amino acid of the invention is in the L form, unless otherwise indicated.

5.2. Methods of Treatment or Prevention

Although not intending to be bound by any particular theory of operation, the present invention is based, in part, on the discovery that certain populations of obese or overweight individuals have undesirable levels of one or more satiety factors and the subpopulation of obese or overweight individuals who have undesirable levels of one or more satiety factors can be positive responders to therapeutic methods for treating obesity or overweight comprising administration of an agonist or antagonist of one or more satiety factors. It is further discovered that that a subpopulation of individuals suffering from conditions such as metabolic disorders or lipid related disorders who have undesirable levels of one or more satiety factors are also positive responders to therapeutic methods comprising administration of an agonist or antagonist of one or more satiety factors.

Accordingly, the present invention provides methods of treating an undesirable level of a satiety factor in a subject in need thereof. In certain embodiments, the methods comprise the step of administering to the subject an amount of an agonist or antagonist of said satiety factor effective for treating the undesirable level of the satiety factor. Whether a subject has an undesirable level of a satiety factor can be determined by any method available to those of skill in the art. Exemplary methods are described herein.

The present invention further provides methods of treating or preventing a disorder or condition associated with an undesirable level of a satiety factor in an subject. The methods comprise the step of administering to the subject an amount of an agonist or antagonist of said satiety factor effective for treating or preventing the disorder or condition.

Exemplary disorders or conditions associated with an undesirable level of a satiety factor include, but are not limited to, overweight, obesity, metabolic disorders, hypertension, lipid related disorders, anorexia and type II diabetes.

In certain embodiments, the disorder or condition associated with an undesirable level of a satiety factor is overweight. In another embodiment, the disorder or condition associated with an undesirable level of a satiety factor is obesity. In certain embodiments, the disorder or condition associated with an undesirable level of a satiety factor is a metabolic disorder. In certain embodiments, the disorder or condition associated with an undesirable level of a satiety factor is a lipid related disorder. In certain embodiments, the disorder or condition associated with an undesirable level of a satiety factor is type II diabetes. In certain embodiments, the disorder or condition associated with an undesirable level of a satiety factor is hypertension. In certain embodiments, the disorder or condition associated with an undesirable level of a satiety factor is anorexia.

In one aspect, the present invention provides methods of reducing appetite in a subject with an undesirable level of a satiety factor in need thereof. The methods comprise the step of administering to said subject an amount of an agonist or antagonist of said satiety factor effective for reducing appetite.

In another aspect, the present invention provides methods of reducing food intake in a subject with an undesirable level of a satiety factor in need thereof. The methods comprise the step of administering to said subject an amount of an agonist or antagonist of said satiety factor effective for reducing food intake.

In another aspect, the present invention provides methods of reducing fat intake in a subject with an undesirable level of a satiety factor in need thereof. The methods comprise the step of administering to said subject an amount of an agonist or antagonist of said satiety factor effective for reducing fat intake.

In another aspect, the present invention provides methods of reducing carbohydrate intake in a subject with an undesirable level of a satiety factor in need thereof. The methods comprise the step of administering to said subject an amount of an agonist or antagonist of said satiety factor effective for reducing carbohydrate intake.

In another aspect, the present invention provides methods of reducing protein intake in a subject with an undesirable level of a satiety factor in need thereof. The methods comprise the step of administering to said subject an amount of an agonist or antagonist of said satiety factor effective for reducing protein intake.

In another aspect, the present invention provides methods of reducing body weight or stimulating weight loss in a subject with an undesirable level of a satiety factor. The methods comprise the step of administering to said subject an amount of an agonist or antagonist of said satiety factor effective for reducing body weight or stimulating weight loss. The term “weight loss” refers to a detectable decrease of body mass in a subject compared to the mass of the subject at a previous time.

In another aspect, the present invention provides methods of reducing body fat in a subject with an undesirable level of a satiety factor in need thereof. The methods comprise the step of administering to said subject an amount of an agonist or antagonist of said satiety factor effective for reducing body fat.

It should be recognized that the methods of the invention encompass the treatment of obesity, methods of reducing appetite, methods of reducing food intake, methods of reducing fat intake, methods of reducing protein intake and methods of reducing carbohydrate intake by selecting patients with a greater likelihood of responding to therapy and administering a therapeutic thereto. Those more likely to respond are identified or selected by measuring or otherwise knowing their levels of one or more endogenous satiety factors.

5.2.1 Subjects

In certain embodiments of the invention, the subject is an animal, preferably a mammal, more preferably a non-human primate. In the most preferred embodiments, the subject is a human. The subject can be a male or female subject.

The methods of the invention can be used for selecting a subject for therapy with an agonist or antagonist of a satiety factor in any subject. Particularly useful subjects include those that have an undesirable level of a satiety factor. Whether a subject has an undesirable level of a satiety factor can be determined by any methods available to those of skill in the art. Exemplary methods are described below.

In certain embodiments, the subject is at risk for a disorder or condition associated with an undesirable level of a satiety factor including, but not limited to, overweight, obesity, metabolic disorders, hypertension, lipid related disorders, anorexia and type II diabetes. In some embodiments, the subject is at risk for overweight or obesity. In some embodiments, the subject is at risk for a metabolic disorder. In some embodiments, the subject is at risk for hypertension. In some embodiments, the subject is at risk for a lipid related disorder. In some embodiments, the subject is at risk for anorexia. In some embodiments, the subject is at risk for type II diabetes.

In certain embodiments, the subject is not healthy. In some embodiments, the subject has diabetes, gastrointestinal and/or cardiovascular diseases. In some embodiments, the subject has or suffers from a disorder or condition associated with an undesirable level of a satiety factor including, but not limited to, overweight, obesity, metabolic disorders, hypertension, lipid related disorders, anorexia and type II diabetes. In some embodiments, the subject has a metabolic disorder. In some embodiments, the subject suffers from hypertension. In some embodiments, the subject has a lipid related disorder. In some embodiments, the subject has type II diabetes. In some embodiments, the subject has abnormal glucose levels. In some embodiments, the subject has anorexia.

In some embodiments, the subject is overweight. In particular embodiments, the subject is a human and has a BMI of 25 kg/m² or greater. In some embodiments, the subject is a human and has a BMI between 25 kg/m² and 30 kg/m². In some embodiments, the subject is obese. In some embodiments, the subject is a human and has a BMI of 30 kg/m² or greater. In some embodiments, the subject is a human and has a BMI between 30 kg/m² and 35 kg/m². In some embodiments, the subject is a human and has a BMI of 35 kg/m² or greater. In some embodiments, the subject is a human and has a BMI of 40 kg/m² or greater. In some embodiments, the subject weighs more than 120% of the normal weight for its age and height. In some embodiments, the subject is a human and weighs more than 96 kg.

In some embodiments, the subject is a human and has a waist circumference greater than 1.02 m. In some embodiments, the subject is a human and has a hip circumference greater than 1.06 m. In some embodiments, the subject is a human and has a waist:hip ratio greater than 0.98. In some embodiments, the subject is a human and has more than 40.9% body fat.

In some embodiments, the subject is within the normal weight range. In the context of this invention, the subject with normal weight include those subjects that, for any reason according to the judgment of a practitioner of the art, are in need of treatment with an agonist or antagonist of a satiety factor. In some embodiments, the subject is a human and has a BMI of 25 kg/m² or less. In some embodiments, the subject is a human and has a BMI of 22 kg/m² or less. In some embodiments, the subject is a human and has a BMI between about 20 kg/m² and about 25 kg/m². In some embodiments, the subject is a human and has a BMI of 20 kg/m² or less. Such a subject could have an undesirable level of a satiety factor with his or her weight maintained, for example, by a condition, such as bulimia.

In some embodiments, the subject has not previously undergone any treatment for a disorder or condition associated with an undesirable level of a satiety factor. In other embodiments, the subject has previously undergone or is now undergoing treatment for a disorder or condition associated with an undesirable level of a satiety factor. In certain embodiments, the subject has previously undergone or is now undergoing treatment with an agonist or antagonist of a satiety factor for such. In certain embodiments, the subject has previously undergone or is now undergoing treatment other than an agonist or antagonist of a satiety factor.

In some embodiments, the subject has abnormal glucose levels. In particular embodiments, the subject has a high blood glucose level. In some embodiments, the subject has diabetes. In certain embodiments, the subject has type II diabetes. In other embodiments, the subject does not have diabetes.

In some embodiments, the subject is below 21 years old. In some embodiments, the subject is below 15 years old. In other embodiments, the subject is more than 49 years old. In some embodiments, the subject is more than 15, 25, 35, 40, 45, 50, 55, or 65 years old.

In some embodiments, the subject exercises regularly. In other embodiments, the subject does not exercise regularly.

5.2.2 The Satiety Factors of the Present Invention

The satiety factor of the present invention can be any molecule that is, or should be endogenously produced by a subject and is capable of regulating appetite, food intake, energy intake or expenditure or satiety signal. In some embodiments, the satiety factor is a peptide. In such embodiments, the peptide satiety factor can be a gut peptide or a non-gut peptide. In other embodiments, the satiety factor is not a peptide.

Satiety factors of the present invention include but are not limited to adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β-endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), melanocyte stimulating hormone (MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, prolactin-releasing peptide, pro-opiomelanocortin (POMC), protoporphyrin, RFU 43 (an RFU amide peptide, 26Rfa), somatostatin, thyrotropin-releasing hormone (TRH), urocortin, and vasopressin, which are described in detail in Bray G A, 1995, Obesity Research 3 (supplement 4):569S-572S; Bays H E, 2004, Obesity Research 12(8):1197-1211, Sahu A, 2004, Endocrinology 145(6); 2613-20, the contents of which are incorporated by reference in their entireties.

In certain embodiments, the satiety factor is a peptide. In some embodiments, the satiety factor is a gut peptide, i.e., a peptide known in the art that is, or should be, produced endogenously by the gastrointestinal tract, such as, for example, stomach, pancreas, intestine or colon, and is capable of regulating appetite, food intake, energy intake, energy expenditure, or satiety. Suitable gut peptides include but are not limited to amylin, bombesin or bombesin-like peptide, cholecystokinin, enterostatin, ghrelin, insulin, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, pancreatic polypeptide and peptide YY. In some embodiments, the gut peptide is enterostatin. In some embodiments, the gut peptide is other than enterostatin

In some embodiments, the satiety factor is amylin. Amylin, a.k.a., islet amyloid polypeptide, consists of 37 amino acids and is released by the β cells of the pancreas. It has been reported that amylin and pramlintide (a synthetic human amylin analogue) administration led to decreased food intake and sustained weigh loss. See Reda et al., 2002, Obesity Res. 10:1087-91, the content of which is incorporated by reference in its entirety.

In some embodiments, the satiety factor is bombesin or bombesin-like peptide. Bombesin or bombesin-like peptide such as, for example, gastrin-releasing peptide and neuromedin B, are widely distributed in the gastrointestinal tract as well as in the central nervous system. Feed suppression by bombesin or bombesin-like peptide have been reported in a variety of species including human. See Yamamda et al., 2002, Euro. J. Pharm. 440:281-290, the content of which is incorporated by reference in its entirety.

In some embodiments, the satiety factor is cholecystokinin. Cholecystokinin (CCK) is produced in gall bladder, pancreas and stomach, and concentrated in the small intestine. It is released mainly in response to dietary fat and functions to increase satiety and decrease appetite. Studies shown that there is a dose-dependent inhibition of food intake following peripheral administration of CCK in many species including both normal weight and obese human subjects. See Kissileff et al., 1981, Am. J. Clin. Nutr. 34:154-60, the content of which is incorporated by reference in its entirety.

In some embodiments, the satiety factor is enterostatin. In some embodiments, the satiety factor is other than enterostatin. Enterostatin is a 5 amino acid peptide generated by tryptic activation of procolipase in the intestine or stomach to generate lipase. See, e.g., Erlanson-Albertsson et al., 1991, Physiol. Behav. 49:1191-1194, the content of which is incorporated by reference in its entirety. The propeptide enterostatin is believed to reduce dietary fat preference in mammals as demonstrated in rodent studies. See, e.g., Erlanson-Albertsson et al., 1991, Physiol. Behav. 49:1191-1194; Okada et al., 1991, Physiol. Behav. 49:1185-1189; Shargill et al., 1991, Brain Res. 544:137-140, the contents of which are incorporated by reference in their entirety.

In some embodiments, the satiety factor is ghrelin. Ghrelin is a 28 amino acid acylated peptide produced mainly by the stomach and an endogenous ligand for the growth seretagogue receptor (GHS-Rs). See Kojima et al. 1999, Nature 402(6762):656660, the content of which is incorporated by reference in its entirety. Circulating levels of ghrelin rise during fasting and after feeding. See Cummings et al., 2001, Diabetes 50:1714-19, the content of which is incorporated by reference in its entirety. In rodents and persons, ghrelin has been shown to increase body weight by stimulating food intake and reducing fat oxidation. See Druce et al., 2006, Intl. J. Obesity 30:293-96; Druce et al., 2005, Intl. J. Obesity 29:1130-36; Wren et al., 2001, Diabetes 141:4325-28, the contents of which are incorporated by reference in their entirety.

In some embodiments, the satiety factor is obestatin. Obestatin is derived from the same peptide precursor (preproghrelin) of ghrelin. See Zhang et al., 2005, Science 310(5750):985-86, the content of which is incorporated by reference in its entirety. Contrary to the appetite stimulating effects of ghrelin, treatment of rats with obestatin suppressed food intake, inhibited jejunal contraction and decreased body weigh gain. See id.

In some embodiments, the satiety factor is selected from proglucagon-derived peptides including, for example, glucagon, glucagon-like peptide 1 (GLP-1), glucagon-like peptide 2 (GLP-2), oxyntomodulin, glicentin, glicentin-related pancreatic peptide, and major proglucagon fragment.

In some embodiments, the satiety factor is oxyntomodulin. Oxyntomodulin, a 37 amino acid peptide, is a product of the proglucagon gene released post-prandially from the L-cells of the small intestine in proportion to calorie intake. It has been shown to regulate satiety signals and reduce energy intake when administered to rodents and humans. See Cohen et al., 2003, J. Clin. Endocrinol Metab. 88:4696-4701; Wynne et al., 2006 (April 19 issue), Int. J. Obesity, the contents of which are incorporated by reference in their entirety.

In some embodiments, the satiety factor is glucagon-like peptide 1 (GLP-1). Glucagon-like peptide-1 (7-36)-amide (GLP-1) is synthesized in the intestinal L-cells by tissue-specific post-translational processing of the glucagon precursor preproglucagon and is released into the circulation in response to a meal. See Varndell et al., 1985, J. Histochem Cytochem, 33:1080-6, the content of which is incorporated by reference in its entirety. Peripheral administration of GLP-1 in both health and obese subjects can suppress hunger and reduce food intake. See Flint et al., 2001, Int. J. Obes. Relat. Metab. Disord. 25(6):781-92; Gutzwiller et al., 1999, Gut 44(1):81-86, the contents of which are incorporated by reference in their entirety.

In some embodiments, the satiety factor is peptide YY. Peptide YY (PYY), a member of the neuropeptide Y protein (NPY) family, is produced and secreted by endocrine cells lining the small intestine and colon in proportion to the calorie content of a meal. See Pedersen-Bjergarrd et al., 1996, Scand. J. Clin. Lab. Invest. 56:497-503; Adrian et al., 1985, Gastroenterology 89:1070-77; Grandt et al., 1994, Regul. Pept. 51:151-59, the contents of which are incorporated by reference in their entirety. The main form of PYY, both stored and in circulation, is PYY₃₋₃₆, an N-terminally truncated form of the full-length peptide. Acting primarily in the brain, PYY₃₋₃₆ is thought to inhibit release of appetite stimulating hormone NPY and to stimulate release of appetite suppressant alpha-Melanocyte Stimulating Hormone. PYY₃₋₃₆ appears to inhibit appetite by acting directly on the arcuate nucleus in the hypothalamus via the Y2 receptor, a G-protein coupled receptor also recognized by NPY. See Batterham et al., 2002, Nature 418(698):650-4, the content of which is incorporated by reference in its entirety.

It has been reported that obese subjects not only have reduced endogenous level of PYY but also have a impaired meal-induced increase in PYY. See Batterham et al., 2003, N Eng. J. Med. 349(10):941-48, the content of which is incorporated by reference in its entirety. Peripheral injection of PYY₃₋₃₆ in rats and mice inhibits food intake and reduced weigh gain. See Batterham et al., 2002, Nature 418(698):650-4, the content of which is incorporated by reference in its entirety. In addition, intravenous PYY₃₋₃₆ infusion into humans results in a significant food intake reduction and blood plasma levels similar to those in normal subjects after a meal. See Batterham et al., 2003, N Eng. J. Med. 349(10):941-48, the content of which is incorporated by reference in its entirety.

In some embodiments, the satiety factor is pancreatic peptide (PP). Like PYY, pancreatic peptide is a 36-amino acid peptide belonging to the NPY family. It is produced in the pancreatic islets and released in response to food digestion. See Adrian et al., 1976, Gut 17:940-44, the content of which is incorporated by reference in its entirety. In rodents, chronic administration of PP reduces food intake and weight gain in genetically obese mice. See Ueno et al., 1999, Gastroenterology 117:1427-32, the content of which is incorporated by reference in its entirety. In humans, intravenous administration of PP causes a sustained decrease in appetite and food intake. See Batterham et al., 2003, J. Clin. Endocrinol Metab. 88:3989-92, the content of which is incorporated by reference in its entirety.

In certain embodiments, the satiety factor is a non-gut peptide, i.e., a peptide known in the art that is, or should be, produced endogenously by an organ or tissue other than the gastrointestinal tract, such as, for example, the brain, liver or adipose tissue, and capable of regulating appetite, food intake, energy intake, energy expenditure, or satiety.

In some embodiments, the satiety factor is adiponectin, which can increase the sensitivity of the hepatocyte to insulin, such as described in Saltiel A R 2001, Nat. Med. (8):887-8, the contents of which are incorporated by reference in their entireties.

In some embodiments, the satiety factor is agouti-related protein (AGRP), present in the hypothalamus, the levels of which are elevated in obese males such as described in Sahu A, 2004, Endocrinology 145(6); 2613-20, Katsuki et al., 2001, J. Clin. Endocr. Metab. 86: 1921-1924, the contents of which are incorporated by reference in their entireties.

In some embodiments, the satiety factor is apolipoprotein A-IV, which can produce a dose dependent reduction of food intake in animals, such as described in Bray G A, 1995, Obesity Research 3 (supplement 4):569S-572S, Fujimoto et al., 1993, J. Clin. Invest. 91 (suppl 4): 1830-33, the contents of which are incorporated by reference in their entireties.

In some embodiments, the satiety factor is beacon, which is expressed in hypothalamus and can stimulate food intake and weight gain in animals in a dose-dependent manner, such as described in Collier G R et al. 2000, Diabetes 49:1766-1771, Brailoiu G C et al., 2002, Neurosci Lett 317(3): 166-8, the contents of which are incorporated by reference in their entireties.

In some embodiments, the satiety factor is brain derived neural factor (BDNF) such as described in Sahu A, 2004, Endocrinology 145(6); 2613-20, the contents of which are incorporated by reference in their entireties.

In some embodiments, the satiety factor is calcitonin-gene related peptide (CGRP), which has been shown to decrease food intake in both obese and non-obese animals, such as described in Bray G A, 1995, Obesity Research 3 (supplement 4):569S-572S, Morley et al. 1982, Peptides 3:17-20, the contents of which are incorporated by reference in their entireties.

In some embodiments, the satiety factor is β casomorphin, which is a seven-amino acid peptide produced during tryptic digestion of casein in the intestinal tract and can increase food intake in animals with a high fat diet and decrease food intake in animals with a low fat diet, such as described in Bray G A, 1995, Obesity Research 3 (supplement 4):569S-572S, Lin et al., 1994, Peptides 15(suppl 5):849-54, the contents of which are incorporated by reference in their entireties.

In some embodiments, the satiety factor is ciliary neurotrophic factor (CNTF), which can cause weigh loss, such as described in Lambert et al., 2001, PNAS 98(8): 4652-57, the contents of which are incorporated by reference in their entireties.

In some embodiments, the satiety factor is cocaine and amphetamine regulated transcript (CART) such as described in Sahu A, 2004, Endocrinology 145(6); 2613-20, the contents of which are incorporated by reference in their entireties.

In some embodiments, the satiety factor is corticotropin-releasing hormone (CRH), which can reduce food intake in animals, such as described in Kristensen P et al. 1998, Nature 393(6680):72-6., the contents of which are incorporated by reference in their entireties.

In some embodiments, the satiety factor is cyclo-his-pro, which is diketopiperzaine and can reduce food intake when administered centrally or peripherally, such as described Bray G A, 1995, Obesity Research 3 (supplement 4):569S-572S, Bray G A, 1992, Am. J. Clin. Nutr. 55:265 S-271S, in the contents of which are incorporated by reference in their entireties.

In some embodiments, the satiety factor is dynorphin, which is produced by the brain and can stimulate food intake in animals such as described in Olszewski et al., 2004, Endocrinology 146(6):2627-2632, the contents of which are incorporated by reference in their entireties.

In some embodiments, the satiety factor is β-endorphin, which has been shown a regulator of energy homeostasis. such as described Appleyard et al. 2003, Endocrinology 144:1753-1760, the contents of which are incorporated by reference in their entireties.

In some embodiments, the satiety factor is galanin or galanin-like peptide (GALP), which stimulate food intake in rats, such as described in Sahu A, 2004, Endocrinology 145(6); 2613-20, Patterson et al., 2006, J. Neuroendocrinol 18(10): 742-747, the contents of which are incorporated by reference in their entireties.

In some embodiments, the satiety factor is growth hormone-releasing hormone (GHRH), which can stimulate food intake and weight gain such as described in Bays H E, 2004, Obesity Research 12(8):1197-1211, Sahu A, 2004, Endocrinology 145(6); 2613-20, the contents of which are incorporated by reference in their entireties.

In some embodiments, the satiety factor is hypocretins/orexins (orexin-A and B), which is identified as ligands for two orphan G-protein coupled orexin receptors-1 and -2, plays a role in feeding and sleep-wakefulness regulation, and can stimulate appetite and food intake in rats, such as described in Sahu A, 2004, Endocrinology 145(6); 2613-20, Adam et al., 2002, Int. J. Obes. 26(2):274-276, Sakurai et al., 1998, Cell 92:573-585, the contents of which are incorporated by reference in their entireties.

In some embodiments, the satiety factor is insulin, or insulin like growth factor I and II (IGF-I and IGF-II), which is a regulator of food intake, such as described in Bray G A, 1995, Obesity Research 3 (supplement 4):569S-572S, Sahu A, 2004, Endocrinology 145(6); 2613-20, the contents of which are incorporated by reference in their entireties.

In some embodiments, the satiety factor is leptin, which can decrease food intake and body weigh, such as described in Sahu A, 2004, Endocrinology 145(6); 2613-20, the contents of which are incorporated by reference in their entireties.

In some embodiments, the satiety factor is melanin concentrating hormone (MCH), which is a cyclic neuropeptide and plays a role in stimulation of feeding behavior in mammals such as described in Shimada et al., 1998, Nature 396: 670-673, the contents of which are incorporated by reference in their entireties.

In some embodiments, the satiety factor is melanocyte stimulating hormone (MSH), which, produced in pituitary, is a ligand for melanocortin receptors and plays a role in the regulation of energy homeostasis, such as described in MacNeil D J et al., 2002, Eur J. Pharmacol. 440(2-3): 141-57, the contents of which are incorporated by reference in their entireties.

In some embodiments, the satiety factor is motilin, which is a polypeptide hormone secreted by Mo cells of the small intestine and can increase the gastrointestinal motility, such as described in Davidson et al., 1999, Phiol. Behav. 66(2):309-315, the contents of which are incorporated by reference in their entireties.

In some embodiments, the satiety factor is nesfatin, which is produced by the brain and can reduce food intake in a dose-dependent manner in animals, such as described in Shinsuke et al., 2006, 443:709-712, the contents of which are incorporated by reference in their entireties.

In some embodiments, the satiety factor is neuromedin B and neuromedin U, which is neuropeptides widely distributed in the gut and central nervous system and is involved in the central control of feeding, such as described in Howard et al. 2000, Nature 406, 70-75, the contents of which are incorporated by reference in their entireties.

In some embodiments, the satiety factor is neuropeptide B (NPB) and (NPW), which is identified as ligands for orphan G protein-coupled receptors and can stimulate food intake in animals, such as described in Shimomura et al. 2002, J. Biol. Chem., 277(39):35826-32, the contents of which are incorporated by reference in their entireties.

In some embodiments, the satiety factor is neuropeptide K (NPK), which can inhibit food intake in animals, such as described in Achapu et al. 1992, Brain Res. Bull 28(2):299-303, Sahu A, 2004, Endocrinology 145(6); 2613-20, the contents of which are incorporated by reference in their entireties.

In some embodiments, the satiety factor is neuropeptide Y (NPY), which can stimulate food intake in animals when injected such as described in Myers et al. 1993, Regul Pept 47, 239-245, the contents of which are incorporated by reference in their entireties.

In some embodiments, the satiety factor is neurotensin (NT), which is a 13-amino-acid neuropeptide and decrease food intake after central administration, such as described in Ohinaka et al., 2004, Peptide 25(12):2135-2138, the contents of which are incorporated by reference in their entireties.

In some embodiments, the satiety factor is oxytocin, which can increase food intake in animals, such as described in Billings et al. 2006, Behav. Brain Res. 171(1)-134-141, the contents of which are incorporated by reference in their entireties.

In some embodiments, the satiety factor is prolactin-releasing peptide, which can increase food intake in animals, such as described in Bray G A, 1995, Obesity Research 3 (supplement 4):569S-572S, Gerardo-Gettens et al., 1989, 256(2pt2):R701-706, the contents of which are incorporated by reference in their entireties.

In some embodiments, the satiety factor is pro-opiomelanocortin (POMC), which is a mediators in the regulation of feeding behavior, insulin levels and body weight, such as described in Boston B A. 2001, J. Pediatr Endocrinol Metab. 14 Suppl 6:1409-16, the contents of which are incorporated by reference in their entireties.

In some embodiments, the satiety factor is protoporphyrin, which is produced during the metabolism of hemoglobin and can decrease food intake and body weight when injected, such as described in Bray G A, 1995, Obesity Research 3 (supplement 4):569S-572S, Galbraith et al., 1991, Am. J. Physiol 261:R1395-41, the contents of which are incorporated by reference in their entireties.

In some embodiments, the satiety factor is QRFP 43 (an RF amide peptide, 26Rfa), which is a regulator of appetite and energy expenditure, such as described in Moriya R, et al. 2006, Endocrinology. 147(6):2916-22, the contents of which are incorporated by reference in their entireties.

In some embodiments, the satiety factor is somatostatin, which has been shown to reduce food intake in animals, such as described in Bays H E, 2004, Obesity Research 12(8):1197-1211, Levine et al., 1982, Pharmacol Biochem Bahav. 16:897-902, the contents of which are incorporated by reference in their entireties.

In some embodiments, the satiety factor is thyrotropin-releasing hormone (TRH), which is a regulator of food intake and energy expenditure, such as described in Al-Arabi et al., 2005, Biomedsci. Instrum. 41: 62-67, the contents of which are incorporated by reference in their entireties.

In some embodiments, the satiety factor is urocortin, which is a high affinity ligand for the type 2 corticotropin-releasing factor and reduces feeding and drinking in rats when infused, such as described in Inoue et al. 2003, J Pharmacol Exp Ther 305(1):385-393, the contents of which are incorporated by reference in their entireties.

In some embodiments, the satiety factor is vasopressin, which has been shown to reduce food intake in animal models, such as described in Bray G A, 1995, Obesity Research 3 (supplement 4):569S-572S, Langhans et al., 1991, Phiol. Behav. 49; 169-176, the contents of which are incorporated by reference in their entireties.

5.2.3 Satiety Factor Agonists and Antagonists

An agonist of a satiety factor can be any agent that mimics the biological activities of, induce similar physiological effects of, or enhances the duration of effects, biological activities or selectivity of a satiety factor. In some embodiments, an agonist of a satiety factor is the satiety factor itself. In other embodiments, an agonist of a satiety factor is an active analogue or derivative, fragment of the satiety factor. An active analogue or derivative, fragment of the satiety factor can be naturally occurring or non-naturally occurring such as exenatide, a non-naturally occurring GLP-1 analogue.

An antagonist of a satiety factor can be any agent that inhibits the biological activities of physiological effect of a satiety factor. An antagonist of a satiety factor can completely or partially inhibit the biological activity of physiological effect of a satiety factor.

In certain embodiments, the satiety factor is amylin and an effective amount of an amylin agonist is administered to a subject in need thereof. In some embodiments, the amylin agonist is amylin. In some embodiments, the amylin agonist is pramlintide, as described in U.S. Pat. No. 5,175,145, 5,686,411, 5,814,600, 5,998,367 or 6,114,2304, the contents of which are hereby incorporated by reference in their entirety.

In certain embodiments, the satiety factor is bombesin or bombesin-like peptide and an effective amount of a bombesin or bombesin-like peptide agonist is administered to a subject in need thereof. In some embodiments, the bombesin or bombesin-like peptide agonist is bombesin or bombesin-like peptide.

In certain embodiments, the satiety factor is cholecystokinin and an effective amount of a cholecystokinin agonist is administered to a subject in need thereof. In some embodiments, the cholecystokinin agonist is cholecystokinin.

In certain embodiments, the satiety factor is enterostatin and an effective amount of an enterostatin is administered to a subject in need thereof. In some embodiments, the enterostatin agonist is enterostatin.

In certain embodiments, the satiety factor is ghrelin and an effective amount of a ghrelin antagonist is administered to a subject in need thereof. In some embodiments, the ghrelin antagonist is as described in U.S. Pat. Pub. Nos. 20050201938, 20050080007, 20040186181 or 20020187938, the contents of which are hereby incorporated by reference in their entirety.

In certain embodiments, the satiety factor is GLP-1 and an effective amount of a GLP-1 agonist is administered to a subject in need thereof. In some embodiments, the GLP-1 agonist is GLP-1. In some embodiments, the GLP-1 agonist is exendin or its analogue as described in U.S. Pat. No. 6,872,700 or 6,989,366, the contents of which are hereby incorporated by reference in their entirety. In some embodiments, the GLP-1 agonist is BYETTA®. In some embodiments, the GLP-1 agonist is an inhibitor of the enzyme dipeptidyl pepdiase IV (DPP IV), which inactivates GLP-1

In certain embodiments, the satiety factor is obestatin and an effective amount of an obestatin agonist is administered to a subject in need thereof. In some embodiments, the obestatin agonist is obestatin.

In certain embodiments, the satiety factor is oxyntomodulin and an effective amount of an oxyntomodulin agonist is administered to a subject in need thereof. In some embodiments, the oxyntomodulin agonist is oxyntomodulin.

In certain embodiments, the satiety factor is peptide YY and an effective amount of a peptide YY agonist is administered to a subject in need thereof. In some embodiments, the peptide YY agonist is peptide YY₃₋₃₆.

In certain embodiments, the satiety factor is pancreatic peptide and an effective amount of a pancreatic peptide agonist is administered to a subject in need thereof. In some embodiments, the pancreatic peptide agonist is pancreatic peptide.

An agonist of a satiety factor can also act through indirect action. For example, an agonist of a glucagon-like peptide 1 (GLP-1), can be an inhibitor of the enzyme dipeptidyl pepdiase IV (DPP IV), which inactivates GLP-1.

In certain embodiments, an agonist of adiponectin, amylin, apolipoprotein A-IV, bombesin or bombesin like peptide, brain derived neural factor (BDNF), calcitonin-gene related peptide (CGRP), cholecystokinin (CCK), ciliary neurotrophic factor (CNTF), cocaine and amphetamine regulated transcript (CART), corticotropin-releasing hormone (CRH), cyclo-his-pro, enterostatin, insulin, insulin like growth factor I and II (IGF-I and IGF-II), leptin, a melanocyte stimulating hormone (α-MSH), motilin, nesfatin, neuromedin B and neuromedin U, neuropeptide b (NPB) and (NPW), neuropeptide K (NPK), neurotensin (NT), obestatin, oxytocin, pancreatic peptide, peptide YY, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, pro-opiomelanocortin (POMC), protoporphyrin, QRFP 43 (an RF amide peptide, 26Rfa), somatostatin, thyrotropin-releasing hormone (TRH), urocortin, or vasopressin is administered.

In certain embodiments, an antagonist of agouti-related protein (AGRP), beacon, β casomorphin, dynorphin, endorphin, galanin, galanin-like peptide (GALP), ghrelin, growth hormone-releasing hormone (GHRH), hypocretins/orexins, melanin concentrating hormone (MCH), neuropeptide Y (NPY), prolactin-releasing peptide, or QRFP 43 (an RF amide peptide, 26Rfa), is administered.

An agonist or antagonist of said satiety factor can be administered by any route known to those of skill in the art, including but not limited to oral, intranasal, intrapulmonary, intravenous, subcutaneous, transdermal, intragastric, intraperitoneal, intracerebroventricular and rectal.

Satiety factor agonists and antagonists can be prepared, formulated and administered to a subject by any methods apparent to those of skill in the art as described below. Of course, pharmaceutically acceptable preparations, formulations and administrations are preferred.

5.2.4 Combination Therapy

In certain embodiments, the methods of the present invention can be used in combination with a second therapy. The second therapy can be any therapy for any condition or disorder known to those of skill in the art. In certain embodiments, the condition or disorder is selected from the group consisting of overweight, obesity, metabolic disorders, hypertension, lipid related disorders, anorexia and type II diabetes.

In certain embodiments, the second therapy can be administered according to a technique deemed suitable to the practitioner in the art.

In certain embodiments, the second therapy is administration of a satiety factor. The satiety factor can be any satiety factor known to those of skill in the art, including those described herein. In certain embodiments, the second therapy satiety factor can be administered according to a technique deemed suitable to the practitioner of skill in the art. In certain embodiments, the second therapy can be administered according to a method described herein, i.e. a method of selecting a subject with an undesirable level of a satiety factor for treatment and administering to the subject an agonist or antagonist of said satiety factor in an amount effective for treating or preventing the disorder or condition.

In certain embodiments, the second therapy is an agonist or antagonist of a non-gut peptide satiety factor. In certain embodiments, the second therapy is a non-gut peptide satiety factor. Examples include adiponectin, agouti-related protein (AGRP), apolipoprotein A-IV, beacon, brain derived neural factor (BDNF), calcitonin-gene related peptide (CGRP), β casomorphin, ciliary neurotrophic factor (CNTF), cocaine and amphetamine regulated transcript (CART), corticotropin-releasing hormone (CRH), cyclo-his-pro, dynorphin, β-endorphin, galanin, galanin-like peptide (GALP), growth hormone-releasing hormone (GHRH), hypocretins/orexins, insulin, insulin like growth factor I and II (IGF-I and IGF-II), leptin, melanin concentrating hormone (MCH), melanocyte stimulating hormone (MSH), motilin, nesfatin, neuromedin B and neuromedin U, neuropeptide b (NPB) and (NPW), neuropeptide K (NPK), neuropeptide Y (NPY), neurotensin (NT), oxytocin, proglucagon-derived peptides including, for example, glucagon, glucagon-like peptide 1 (GLP-1), prolactin-releasing peptide, pro-opiomelanocortin (PMOC), protoporphyrin, QRFP 43 (an RF amide peptide, 26Rfa), somatostatin, thyrotropin-releasing hormone (TRH), urocortin, and vasopressin.

In certain embodiments, the second therapy is a gut peptide satiety factor. Examples include amylin, bombesin or bombesin-like peptide, cholecystokinin, enterostatin, ghrelin, proglucagon-derived peptides including, for example, glucagon, glucagon-like peptide 1 (GLP-1), glucagon-like peptide 2 (GLP-2), oxyntomodulin, glicentin, glicentin-related pancreatic peptide and major proglucagon fragment, obestatin, oxyntomodulin, pancreatic polypeptide and peptide YY.

The use of combination therapy does not limit the order in which agents or treatments are administered to a subject in the methods provided. For example, the agents of the combination therapy can be administered concurrently, sequentially in any order or cyclically to a subject. In some embodiments, two components of a combination therapy are administered concurrently to a subject.

Components of combination therapy can be administered to a subject in the same pharmaceutical composition. Alternatively, components of combination therapies can be administered to a subject in separate pharmaceutical compositions, and these separate compositions may be administered by the same or by different routes of administration, including, for example, oral, parenteral, or topical.

In preferable embodiments, the other therapies are administered to subject according to their respective standard or art-recognized doses and dosing schedules.

In some embodiments, the other therapies are selected for its additive effects with satiety factor agonists or antagonists for the treatment or prevention of a disorder or condition associated with an undesirable level of a satiety factor.

In some embodiments, the other therapies are selected for its synergistic effects with satiety factor agonists or antagonists for the treatment or prevention of a disorder or condition associated with an undesirable level of a satiety factor.

Examples of therapies that can be used in the methods provided herein, include but are not limited to diet, exercise, life style and behavior modification, sympathomimetic adrenergic agents such as amphetamines (dextroamphetamine), phentermine, benzphetamine, phendimetrazine, mazindol, diethylpropion, phenylpropanolamine, serotonin (5-HT) reuptake inhibitors such as sibutramine, gastrointestinal lipases such as orlistat. See Obesity Research 12(8):1197-1211, the contents of which are incorporated by reference in their entireties.

5.3. Methods of Identifying Subjects for Treatment with a Satiety Factor Agonist or Antagonist

The present invention is based, in part, on the discovery that treatments of obesity and related diseases with an agonist or antagonist of a satiety factor can be effective in subjects that are responsive to satiety factor treatment, and subjects that are responsive to satiety factor treatment include those that have an undesirable endogenous level of a satiety factor.

5.3.1 Determining the Level of Satiety Factor

The endogenous level of satiety factor in a subject can be determined by any method available to those of skill in the art. It can be determined directly or indirectly. In some embodiments, it is determined from measuring the amount of satiety factor in a sample from a subject. In other embodiments, it is determined from measuring the activity of a precursor of a satiety factor in a sample from a subject. For instance, the level of enterostatin can be determined from measuring the activity of procolipase in a sample from a subject. In addition to the total endogenous level of satiety factor, the endogenous level of an active form of the satiety factor in a subject can determined. For example, both the total level of ghrelin, and the level of acylated ghrelin, are determined in certain embodiments.

In certain embodiments of the invention, the method of determining the amount of satiety factor is not critical. Accordingly, the present invention provides methods for selecting a subject for treatment with a satiety factor agonist or antagonist that comprise the single step of determining whether a subject is suitable for treatment based on the amount of satiety factor in a sample from the subject.

The amount of satiety factor can be determined by one practicing a method of the invention in any manner whatsoever. Exemplary techniques are described herein.

The amount of satiety factor can be determined from any sample from the subject, which can be, by way of example and not of limitation, a blood sample, a plasma sample, a saliva sample, a serum sample, a sputum sample, a urine sample, a stool sample, a cell sample, a cellular extract sample, a tissue biopsy sample or any sample that may be obtained from a subject using techniques well known to those of skill in the art. The precise sample that is taken from the subject may vary, but the sampling preferably is minimally invasive and is easily performed by conventional techniques.

The sample can be processed or purified according to the judgment of those of skill in the art based on, for example, the type of sample used and the measurement technique. Particularly useful processing steps are precipitation, centrifugation, filtration and/or chromatography.

5.3.2 Measuring the Amount of Satiety Factor

The amount of satiety factor in a sample from a subject can be determined by any method known to those of skill in the art without limitation. For example, it can be determined by spectrometry, chromatography, immunoassay or electrophoresis. In some embodiments, the amount of satiety factor is determined by immunoassay. In some preferred embodiments, the amount of satiety factor is determined by ELISA.

In preferred embodiments, the amount of amylin is determined as described by Ludvik et al., 1991, Diabetes 40(12):1615-19, the content of which is incorporated by reference in its entirety.

In preferred embodiments, the amount of enterostatin is determined as described by Imamura et al, 1998, Peptides, 19:8, 1385-1391; Bowyer et al., 1991, Clinica. Chimica. Acta. 200:137-152; Mizuma et al., 1995, Biochemical & Biophysical Research Communications 1995, 215(1): 227-234; or Zhao et al., 2001, Fresenius J. Anal. Chem. 269:220-224, the contents of which are incorporated by reference in its entirety.

In preferred embodiments, the amount of GLP-1 is determined as described by Kreymann et al., 1987, Gut 2(8571):1300-04; Verdich et al., 2001, Int. J. Obesity 25:1206-14; or Feinle et al., 2002, Peptides 23:1491-95, the contents of which are incorporated by reference in their entirety.

In preferred embodiments, the amount of ghrelin is determined as described by Druce et al., 2005, Intl. J. Obesity 29:1130-36; Akamizu et al, 2005, J. Clin. Endodrinol. Metab. 90(1):6-9 (for acylated and deacylated ghrelin); or Tschop et al., 2001, Diabetes, 50:707-09, the contents of which are incorporated by reference in their entirety.

In preferred embodiments, the amount of PYY is determined as described by, Batterham et al., 2003, N Eng. J. Med. 349(10):941-8; Adrian T E et al, 1987, Surgery 101(6):715-19; Savage et al., 1987, Gut 28(2): 166-70; or Fuessl et al., 1988, Klin Wochenschr 66(19): 985-89, the contents of which are incorporated by reference in their entirety.

In preferred embodiments, the amount of pancreatic polypeptide is determined as described by Berntson et al., 1993, Peptides. 14(3):497-503; Polak et al., 1976, Lancet. 1(7955):328-30; or Adrian et al., 1976, Gut 17(5):393-94; the content of which are incorporated by reference in its entirety.

Standard techniques for determining the amount of a peptide or a peptide of interest present in a sample may be utilized for determining the amount of satiety factor in a sample. For example, standard techniques can be employed using, e.g., immunoassays such as, for example Western blot, immunoprecipitation followed by sodium dodecyl sulfate polyacrylamide gel electrophoresis, (SDS-PAGE), immunocytochemistry and the like to determine the amount of protein or proteins of interest present in a sample. One exemplary agent for detecting a protein of interest is an antibody capable of specifically binding to a peptide of interest, preferably an antibody detectably labeled, either directly or indirectly.

For such detection methods, if desired the satiety factor from the sample can easily be isolated using techniques which are well known to those of skill in the art. Those methods can, for example, be such as those described in Harlow and Lane, 1988, Antibodies. A Laboratory Manual. Cold Spring Harbor Laboratory Press (Cold Spring Harbor, N.Y.), which is incorporated by reference herein in its entirety.

In certain embodiments, methods of detecting the amount of satiety factor in a sample involve detection via interaction with an antibody that is capable of specifically binding a satiety factor. Antibodies can be obtained from commercial sources or can be generated utilizing standard techniques well known to those of skill in the art. In specific embodiments, antibodies can be polyclonal, or more preferably, monoclonal. An intact antibody, or an antibody fragment (e.g., scFv, Fab or F(ab′)₂) can, for example, be used. Exemplary immunoassays are described below.

In some embodiments, a protein chip assay (See, e.g., Zhu & Snyder, 2003, Curr. Opin. Chem. Biol. 7:55-63; Mitchell, 2002, Nature Biotechnology 20:225-229) is used to measure amounts for the biomarkers in the biomarker profile. See also, for example, Lin, 2004, Modern Pathology, 1-9; Li, 2004, Journal of Urology 171, 1782-1787; Wadsworth, 2004, Clinical Cancer Research, 10, 1625-1632; Prieto, 2003, Journal of Liquid Chromatography & Related Technologies 26, 2315-2328; Coombes, 2003, Clinical Chemistry 49, 1615-1623; Mian, 2003, Proteomics 3, 1725-1737; Lehre et al., 2003, BJU International 92, 223-225; and Diamond, 2003, Journal of the American Society for Mass Spectrometry 14, 760-765, which are hereby incorporated by reference in their entireties. Particularly useful in certain embodiments of the invention are antibody chips that facilitate detection by MALDI or SELDI (See, e.g., Wang, et al., 2001, Int'l. J. of Cancer 92:871-876; Figeys, 2002, Proteomics 2:373-382; Sonksen et al., 1998, Anal. Chem. 70:2731-6; Glökler, & Angenendt, 2003, J. Chromatography B, 797:229-240; the contents of which are hereby incorporated by reference in their entireties).

In certain embodiments, antibodies, or fragments of antibodies, specific for satiety factor can be used to determine the amount of satiety factor in a sample. This can be accomplished, for example, by immunofluorescence techniques. Antibodies (or fragments thereof) can, additionally, be employed histologically, as in immunofluorescence or immunoelectron microscopy, for in situ determination of a satiety factor.

Immunoassays typically comprise incubating a sample of a detectably labeled antibody capable of identifying a satiety factor, and detecting the bound antibody by any of a number of techniques well-known in the art. Exemplary immunoassays are Western plot, immunoprecipitation followed by sodium dodecyl sulfate polyacrylamide gel electrophoresis (SDS-PAGE), immunocytochemistry and the like to the determine the amount of a peptide in a sample.

One of the ways in which an antibody specific for satiety factor can be detectably labeled is by linking the same to an enzyme and use in an enzyme immunoassay (EIA) (Voller, 1978, “The Enzyme Linked Immunosorbent Assay (ELISA)”, Diagnostic Horizons 2:1-7, Microbiological Associates Quarterly Publication, Walkersville, Md.; Voller et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Igaku Shoin, Tokyo, each of which is hereby incorporated by reference in its entirety). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection can also be accomplished by visual comparison of the extent of enzymatic reaction of a substrate in comparison with similarly prepared standards.

Measurement can also be accomplished using any of a variety of other immunoassays. For example, by radioactively labeling the antibodies or antibody fragments, it is possible to detect a biomarker through the use of a radioimmunoassay (RIA) (See, for example, Weintraub, B., Principles of Radioimmunoassays, Seventh Training Course on Radioligand Assay Techniques, The Endocrine Society, March, 1986, which is incorporated by reference herein). The radioactive isotope (e.g., ¹²⁵I, ¹³¹I, ³⁵S or ³H) can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The amount of satiety factor can also, for example, be determined by the use of one or more of the following methods described below. For example, methods may include nuclear magnetic resonance (NMR) spectroscopy, a mass spectrometry method, such as electrospray ionization mass spectrometry (ESI-MS), ESI-MS/MS, ESI-MS/(MS)ⁿ (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APC-MS), APCI-MS/MS, APCI-(MS)ⁿ, atmospheric pressure photoionization mass spectrometry (APPI-MS), APPI-MS/MS, and APPI-(MS)ⁿ. Other mass spectrometry methods may include, inter alia, quadrupole, Fourier transform mass spectrometry (FTMS) and ion trap. Other suitable methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. In one embodiment, the biological sample may be fractionated prior to application of the separation method.

In one embodiment, laser desorption/ionization time-of-flight mass spectrometry is used to determine the amount of a biomarker where the biomarker is a molecule that has been ionized and vaporized off an immobilizing support by incident laser radiation. A variety of laser desorption/ionization techniques are known in the art (See, e.g., Guttman et al., 2001, Anal. Chem. 73:1252-62 and Wei et al., 1999, Nature 399:243-246, which are hereby incorporated by reference).

Laser desorption/ionization time-of-flight mass spectrometry allows the generation of large amounts of information in a relatively short period of time. A biological sample is applied to one of several varieties of a support that binds all of the biomarkers, or a subset thereof, in the sample. Cell lysates or samples are directly applied to these surfaces in volumes as small as 0.5 μL, with or without prior purification or fractionation. The lysates or sample can be concentrated or diluted prior to application onto the support surface. Laser desorption/ionization is then used to generate mass spectra of the sample, or samples, in as little as three hours.

Analysis by liquid chromatography-mass spectrometry produces a mass intensity spectrum, the peaks of which represent various components of the sample, each component having a characteristic mass-to-charge ratio (m/z) and retention time (r.t.). The presence of a peak with the m/z and retention time of a biomarker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation.

In MALDI mass spectrometry (MALDI-MS), various mass analyzers can be used, e.g., magnetic sector/magnetic deflection instruments in single or triple quadrupole mode (MS/MS), Fourier transform and time of flight (TOF), including orthogonal time-of-flight (O-TOF), configurations as is known in the art of mass spectrometry. For the desorption/ionization process, numerous matrix/laser combinations can be used. Ion trap and reflectron configurations also can be employed.

Electrospray ionization mass spectrometry (ESI-MS) is broadly applicable for analysis of macromolecules, including proteins, nucleic acids and carbohydrates (Fenn et al., 1989, Science 246:64-71; Crain et al., 1998, Curr. Opin. Biotechnol. 9:25-34; Smith et al., 1990, Anal Chem. 62:882-99; Han & Gross, 1994, Proc Natl Acad Sci USA 91: 10635-10639). Electrospray techniques have been used to separate and measure biomarkers like those of formula I and formula Ia (See Petkovic et al., 2001, Anal Biochem. 289(2):202-16; Pulfer & Murphy, 2003, Mass Spec Rev 22:332-364; Han & Gross, 1995, J. Amer. Soc. Mass Spec. 6:1202-1210; the contents of which are hereby incorporated by reference in their entireties).

For proteins or peptides, Vorm, O. et al., Anal. Chem. 66:3281-3287 (1994); and Vorm and Mann, J. Am. Soc. Mass. Spectrom. 5:955-958 (1994)), for example, provide additional guidance on mass spectral analysis of such molecules and are incorporated by reference in their entirety. The contents of these publications are hereby incorporated by reference in their entireties.

5.3.3 Selecting Subjects for the Treatment with Satiety Factor

Whether a subject has an undesirable level of a satiety factor can be determined by any method available to those of skill in the art. Exemplary methods are described herein.

In certain embodiments, subjects with low level of a satiety factor or subjects with a satiety factor deficiency are selected for treatment. The subject is selected for treatment when the amount of satiety factor in the sample of the subject is less than a normal satiety factor value. In some embodiments, a subject that does not express or secrete an amount of satiety factor detectable using techniques available in the art is selected. In other embodiments, the subject that does not express or secrete any of the satiety factor is selected. In preferred embodiments, a subject is selected when the subject expresses or secretes a lower amount of satiety factor after a meal than a control subject does. The low level of the satiety factor in a subject can be due to any cause known in the art. For example, it may be due to low level of expression or secretion of satiety factor, or due to inadequate activation of the satiety factor in the intestine or stomach. It can also be a consequence of excessive proteolytic activity, such as excessive protease activity that can hydrolyze the satiety factor.

In certain embodiments, subjects with high level of a satiety factor or subjects with satiety factor overproduction are selected for treatment. The subject is selected for treatment when the amount of a satiety factor in the sample of the subject is more than a normal satiety factor value. In preferred embodiments, a subject is selected when the subject expresses or secretes a higher amount of a satiety factor in fasting state than a control subject does. The high level of the satiety factor in a subject can be due to any cause known in the art. For example, it may be due to high level of expression or secretion of the satiety factor.

In certain embodiments, the selection can be based on the amount of a satiety factor in a sample of the subject and a normal satiety factor value. Normal satiety factor values are described in the section below. In some embodiments, if the amount of a satiety factor in the test subject is below, or substantially below, the normal satiety factor value, the test subject is selected for treatment with the satiety factor. In some embodiments, the subject is selected when the amount of a satiety factor in the sample from the subject is less than a normal satiety factor value. In other embodiments, a subject is selected when the amount of a satiety factor in the sample from the subject is less than 95%, 90%, 85%, 80%, 75%, 70%, 65%, 60%, 55%, 50%, 45%, 40%, 35%, 30%, 25%, 20%, 15%, 10%, 5%, 2% or 1% of a normal satiety factor value.

In other embodiments, if the amount of satiety factor in the test subject is above, or substantially above, the normal satiety factor value, the test subject is selected for treatment with the satiety factor. In some embodiments, the subject is selected when the amount of a satiety factor in the sample from the subject is more than a normal satiety factor value. In other embodiments, a subject is selected when the amount of satiety factor in the sample from the subject is about 110%, 120%, 130%, 140%, 150%, 160%, 170%, 180%, 190%, 200%, 250%, 300%, 400%, 500%, 600%, 700%, 800%, 900%, 1000% or 1500% of a normal satiety factor value.

In certain embodiments, one or more samples taken at a single point in time from the subject are used to make the selection. In some embodiments, only a single sample at a single time point is taken. In other embodiments, a plurality of samples taken at a different time points from the subject are taken. A plurality of samples can be the same or different sample types. In particular embodiments, both a blood sample and a urine sample are taken from the subject to make the selection. When a plurality of the same type of samples is used, the evaluation can be based on any statistical technique know to those of skill in the art, such as ANOVA or Chi squared test.

When a sample at a single time point is used, the sample can be obtained from the subject when the subject is fasted overnight, when the subject is fed, or about 0.5, 1.0, 1.5, 2.0, 2.5, 3.0, 3.5 or 4.0 hours after the subject is fed. In some embodiments, the subject is fed with a regular meal, a high-fat meal or a high carbohydrate meal.

In certain embodiments, the meal comprises carbohydrate. In certain embodiments, the meal comprises protein. In certain embodiments, the meal comprises fat. In certain embodiments, the meal is a challenge with a specific nutrient. In certain embodiments, the meal is challenge with carbohydrate. In certain embodiments, the meal is challenge with protein. In certain embodiments, the meal is challenge with fat.

In certain embodiments, a plurality of samples taken at different points in time from the subject are used to make the selection. The times can be separated according to the judgment of those of skill in the art of skill in the art. In some embodiments, these samples are obtained from the subject either on a daily basis, or alternatively more frequently, e.g., every 4, 6, 8, or 12 hours.

In some embodiments, a plurality of samples taken at different time points is for purpose of repeated measurement. In such embodiments, the evaluation can be based on any statistical technique know to those of skill in the art, such as ANOVA or Chi squared test. Preferably, the samples are taken from the subject when the subject is under the same or similar feeding conditions according to the judgment of a practitioner of skill. In some embodiments, the samples are taken when the subject is fasted overnight. In some embodiments, the samples are taken when the subject is fed. In some embodiments, the samples are taken about a particular time after the subject is fed. In particular embodiments, all of samples are taken about 0.5, 1.0, 1.5, 2.0, 2.5, 3.0, 3.5 or 4.0 hours after the subject is fed with a meal.

In other embodiments, a plurality of samples taken at different time points from the subject and a change or no change in the amount of satiety factor is evaluated to make the selection. It has been reported that obese subjects not only have altered endogenous level of satiety factors but also have an impaired meal-induced response in the level of satiety factors. See e.g. Verdich et al., 2001, Int. J. Obesity 25:1206-14 (GLP-1); le Roux et al., 2005, J. Clin. End. & Metab. 90(2):1068-71 (ghrelin). Accordingly, the ratio of fed:fast of the amount of satiety factor can be determined and used to select a subject for treatment with an agonist or antagonist of a satiety factor.

In certain embodiments, samples are taken both when the subject is fasted overnight, and when the subject is fed or over the course of one, two, or three hours after the subject is fed with a meal, and the ratio of fed:fast of the amount of a satiety factor from the subject is calculated. In certain embodiments, the satiety factor levels are measured continuously for one, two or three hours following the meal. In some embodiments, a subject is selected when the ratio of fast:fed is reduced relative to a normal satiety factor fast:fed ratio. In another embodiment, a subject is selected when the ratio of fast:fed is less than 95%, 90%, 85%, 80%, 75%, 70%, 65%, 60%, 55%, 50%, 45%, 40%, 35%, 30%, 25%, 20%, 15%, 10%, 5%, 2% or 1% of a normal satiety factor fast:fed ratio. In some embodiments, a subject is selected when the ratio of fast:fed is increased relative to a normal satiety factor fast:fed ratio. In another embodiment, a subject is selected when the ratio of fast:fed is about 110%, 120%, 130%, 140%, 150%, 160%, 170%, 180%, 190%, 200%, 250%, 300%, 400%, 500%, 600%, 700%, 800%, 900%, 1000% or 1500% of a normal satiety factor fast:fed ratio. The normal fed:fast ratio of a satiety factor is described below.

In certain embodiments, the endogenous levels of a plurality of satiety factors are determined and used to select a subject for the treatment with an agonist or antagonist of a satiety factor. For example, a satiety factor profile, including but not limited to amylin, bombesin or bombesin-like peptide, cholecystokinin, enterostatin, ghrelin, glucagon-like peptide 1, obestatin, oxyntomodulin, pancreatic polypeptide and peptide YY, is determined and used to select a subject for the treatment with an agonist or antagonist of a satiety factor.

In addition to the level of a satiety factor, other parameters or variables can be used in combination with the level of a satiety factor to select a subject for treatment with an agonist or antagonist of a satiety factor. In some embodiments, the blood glucose level of the subject is used. In other embodiments, the body weight or BMI of the subject is used to make the selection. Further, whether a subject is selected for treatment with an agonist or antagonist of a satiety factor may be in accordance with the judgment of those of skill in the art, for instance, based on the blood testing, eletrocardiogram, fasting chemistry panel, CBC, blood pressure, pulse rate, urinalysis, adverse event of the subject in combination with the level of a satiety factor.

In certain embodiments, the amounts of a plurality of satiety factors can be determined in the subject. The subject is selected if one or more of the amounts are undesirable, as described above. If one amount is undesirable, the subject can be administered an agonist or antagonist of the corresponding satiety factor, as described in the methods above. If more than one amount is undesirable, the subject can be administered a combination of agonists or antagonists of the corresponding satiety factors. The combination can be according to any combination of the agonists or antagonists deemed safe and effective by one of skill in the art. The agonists or antagonists can be administered together in a mixture, or in simultaneous but separate dosages, or in cycling dosages or according to any other schedule determined by one of skill in the art.

In certain embodiments, the amounts of a panel of satiety factors can be determined in the subject. The subject is selected if one or more of the amounts are undesirable, as described above. Advantageously, the panel can be used to personally tailor administration of agonists or antagonists to the subject. Each agonist or antagonist can be administered according to methods known to those of skill in the art such as those described herein. The agonists or antagonists can be administered together in a mixture, or in simultaneous dosages, or in cycling dosages or according to any other schedule determined by one of skill in the art. In particular embodiments, a mixture or cocktail of agonists or antagonists can be selected for the subject based on the amounts measured with the panel. For example, if the subject low levels of enterostatin and high levels of ghrelin, both measured according to the methods of the invention, the subject can be administered an agonist of enterostatin and an antagonist of ghrelin in combination.

In certain embodiments, the panel comprises two or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises three or more gut peptides selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises four or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises five or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises six or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises seven or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises eight or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises nine or more satiety factors selected from amylin, bombesin or bombesin-like peptide, cholecystokinin, enterostatin, ghrelin, glucagon-like peptide 1, obestatin, oxyntomodulin, pancreatic polypeptide and peptide YY. In certain embodiments, the panel comprises the satiety factors selected amylin, bombesin or bombesin-like peptide, cholecystokinin, enterostatin, ghrelin, glucagon-like peptide 1, obestatin, oxyntomodulin, pancreatic polypeptide and peptide YY.

5.3.4 Normal Satiety factor Value and Normal Satiety Factor Fed:Fast Ratio

The normal satiety factor value can be, for example, the amount of a satiety factor in a sample from a control subject or a plurality of control subjects. The amount of a satiety factor in a control subject or control subjects can be measured according to techniques known to those of skill in the art including those described herein. Advantageously, in certain embodiments, the amount of a satiety factor in control subject and the amount of a satiety factor in the test subject are obtained by the same technique. Those of skill in the art would understand that a normal satiety factor value may vary for each particular assay, each sample type, and each type of cell-free extract. Those of skill in the art would understand that a normal satiety factor value may vary depending on different species or gender of subjects to be selected. For example, a normal satiety factor value may be higher for a male subject than a female subject of the same species. Accordingly, in preferred embodiments, the satiety factor level for a female subject is compared to the level expected for a female subject; the satiety factor level for a male subject is compared to the level expected for a male subject.

Normal satiety factor values can be determined according to methods described herein or from other sources available to those of skill in the art. In certain embodiments, normal satiety factor values are determined by measuring the values in normal subjects. The subjects should be determined to be normal, for instance not obese or not overweight, by a practitioner of skill in the art. Preferred normal subjects are comparable to treatment subjects where possible, e.g. gender, age, height, etc. The satiety factor value in such a subject can be determined according to methods described herein or according to other methods apparent to those of skill in the art. Normal satiety factor values can also be determined from sources available to those of skill in the art including, for example, literature, clinical trials, available databases and the like. Exemplary, nonlimiting, references providing satiety factor values include Alevizake et al., 2001, Eur. J. Endocrinol. 145:585-9; Schou et al., 2005, J. Clin. Endocrinl. Metabol. 90:4912-4919; Peracci et al., 1999, Scand. J. Gastroenterol. 34:25-28; Teitelbaum et al., 1989, J. Pediatr. Surg. 24:629-633; Zhou et al., 2006, Obesity 14:683-689; Batterham & Bloom, 2003, Ann. N.Y. Acad. Sci. 994:162-168; Kim et al., 2005, J. Clin. Endocrinl. Metabol. 90:6665-6671; Teff et al., 2004, J. Clin. Endocrinl. Metabol. 89:2963-2972; Espelund et al., 2005, J. Clin. Endocrinl. Metabol. 90:2741-2746; le Roux et al., 2006, Ann. Surgery 243:108-114; the contents of each of which are hereby incorporated by reference in their entireties.

The control subject can be a lean subject or a subject with normal weight. When the subject is human, the control subject can be an individual or individuals with normal BMI range of 20-25 kg/m². In certain embodiments, the normal satiety factor amount is from a plurality of control subjects presenting no symptom of the disorder or condition associated with an undesirable level of a satiety factor. The normal satiety factor amount can be calculated according to any suitable statistical method known to those of skill in the art. For instance, the normal satiety factor amount can be based on the statistical mean of the satiety factor amount in samples from control subjects presenting no symptom of the disorder or condition associated with an undesirable level of a satiety factor.

Advantageously, the normal satiety factor value need not be obtained or measured by a practitioner of a method of the invention. Instead, the amount of the normal satiety factor value can be identified by consultation in sources available to those of skill in the art, such as scientific literature, public or private databases, or by reference to the data provided herein.

The normal satiety factor value can be an absolute value, an absolute value with a margin of error or a range of values, as determined by those of skill in the art. In certain embodiments, the selection is made based on a range of normal values for the amount of satiety factor. The range of normal values can be obtained as described herein and made available to a practitioner of the methods of the invention.

In certain embodiments, the normal satiety factor value is a cutoff reference amount. A cutoff reference amount is an absolute value for the normal satiety factor amount. Cutoff reference amounts can be determined using statistical techniques known to those of skill in the art based on control amounts obtained from control subjects. For instance, it can be based on the statistical mean of the amount of satiety factor in samples from control subjects.

The amount of satiety factor in a sample from a subject can be compared with a normal satiety factor value according to any suitable statistical method known to those of skill in the art. In preferred embodiments, two- or three-way analysis of variance (ANOVA) or Chi squared test is used for comparison with repeated measurement.

In certain embodiments, the subject is selected for therapy with an agonist or antagonist of a satiety factor, based on the fed:fast ratio of satiety factor in a sample of the subject and a normal satiety factor fed:fast ratio. The fed:fast ratio of satiety factor is obtained by dividing the amount of satiety factor in a sample from a subject when the subject is fasted overnight by the amount of satiety factor in the sample when the subject is fed or about 0.5, 1, 1.5, 2, 2.5 or 3 hours after the subject is fed. The above description regarding normal satiety factor values also applies to normal satiety factor fed:fast ratios. For instance, the normal satiety factor fed:fast ratio can be, for example, the normal satiety factor fed:fast ratio from a control subject or a plurality of control subjects, and it need not be obtained or measured by a practitioner of a method of the invention. It may vary for each particular assay, each sample type, and each type of cell-free extract. It can be an absolute value, an absolute value with a margin of error, a range of values, or a cutoff reference amount.

Formulation and Route of Administration of Satiety factor Agonists or Antagonists

Satiety factor agonists or antagonists for use in the treatment may be administered to a subject per se, in the form of a pharmaceutical composition, in a form of a co-complex, or in a form of a pharmaceutical composition comprising a co-complex.

Satiety factor agonists or antagonists can be administered by any route according to the judgment of those of skill in the art, including but not limited to oral, intranasal, intrapulmonary, intravenous, subcutaneous, transdermal, intragastric, intraperitoneal, intracerebroventricular and rectal.

In a preferred embodiment, a composition for administration is a pharmaceutical composition or a single unit dosage form. Pharmaceutical compositions and single unit dosage forms can comprise a prophylactically or therapeutically effective amount of one or more prophylactic or therapeutic agents, and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre-gelatinized starch and magnesium stearate.

This invention further encompasses administration of anhydrous pharmaceutical compositions and dosage forms comprising an agonist or antagonist of a satiety factor. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

The invention further encompasses administration of pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition comprising an agonist or antagonist of a satiety factor is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape and type of dosage forms of an agonist or antagonist of a satiety factor will typically vary depending on their use. For example, a dosage form used in the acute treatment of a disorder may contain larger amounts of one or more of an agonist or antagonist of a satiety factor it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions comprising the an agonist or antagonist of a satiety factor are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms for administration in methods of the invention comprise an agonist or antagonist of a satiety factor an agonist or antagonist of a satiety factor an agonist or antagonist of a satiety factor or a co-complex of comprising an agonist or antagonist of a satiety factor, or a pharmaceutically acceptable salt, solvate or hydrate thereof lie within the range of from about 0.001 mg to about 1000 mg or about 0.1 pmol/l to about 100 pmol/I per day, given as a single once-a-day dose in the morning but preferably as divided doses throughout the day taken with food.

5.3.5 Oral Dosage Forms

Pharmaceutical compositions used in the methods of the invention that are suitable for oral administration can be presented as discrete dosage forms, such as, but are not limited to, tablets (e.g., chewable tablets), caplets, capsules and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In preferred embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives and coloring agents. Examples of excipients suitable for use in solid oral dosage forms (e.g., powders, tablets, capsules and caplets) include, but are not limited to, starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.3.6 Transdermal, Topical & Mucosal Dosage Forms

Although solid, anhydrous oral dosage forms are preferred, the present invention also provides administration of an agonist or antagonist of a satiety factor in transdermal, topical, or mucosal dosage forms. Transdermal, topical and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.3.7 Dosage & Frequency of Administration

The amount of an agonist or antagonist of a satiety factor in the methods of the invention which will be effective in the prevention, treatment, management, or amelioration of a disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems.

Exemplary doses of an agonist or antagonist of a satiety factor include milligram or microgram amounts of the agonist or antagonist per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

The satiety factor agonists or antagonists can be administered as a single once-a-day dose or preferably as divided doses throughout a day. In some embodiments, the daily dose is administered twice daily in equally divided doses. In other embodiments, the daily dose is administered three times per day. In particular embodiments, the daily dose is administered three times per day in equally divided doses. In some embodiments, the daily dose is administered three times per day in three divided doses and each dose comprises the satiety factor agonists or antagonists in an amount between about 0.0001-100 mg, about 0.001-10 mg, about 0.01-1 mg, or about 0.001-1000 pmol/l, 0.01-100 pmol/l or 0.1-10 pmol/l. Preferably, the three divided doses of the satiety factor agonists or antagonists are given around three meal times each day. In certain embodiments, the agonist or antagonist can be administered continuously using, for example, a transdermal or osmotic or pump delivery system such as those described above.

The satiety factor agonist or antagonist can be administered at various times. In some embodiments, it is administered to a subject with an undesirable level of a satiety factor when the subject is fasted. In some embodiments, it is administered prior to a meal. In some embodiments, it is administered during a meal. In some embodiments, it is administered after a meal.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with administration of satiety factor agonists or antagonists of the invention are also encompassed by the above described dosage amounts and dose frequency schedules. Further, when a patient is administered multiple dosages of satiety factor agonists or antagonists of the invention, not all of the dosages need be the same. For example, the dosage administered to the patient may be increased to improve the prophylactic or therapeutic effect of the co-complex or it may be decreased to reduce one or more side effects that a particular patient is experiencing.

In certain embodiments, administration of satiety factor agonists or antagonists in the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

In certain embodiments, the methods and compositions can be practiced as a single, one time dose or chronically. By chronic it is meant that the methods and compositions of the invention are practiced more than once to a given individual. For example, chronic administration can be multiple doses of a pharmaceutical composition administered to a subject, on a daily basis, twice daily basis, or more or less frequently, as will be apparent to those of skill in the art. Chronic administration can continue for days, weeks, months or years if appropriate according to the judgment of the practitioner of skill.

In another embodiment, the methods and compositions are practiced acutely. By acute it is meant that the methods and compositions of the invention are practiced in a time period close to or contemporaneous with the onset of an event. For example, acute administration can be a single dose or multiple doses of a pharmaceutical composition administered around the onset of a meal. In some embodiments, the meal is a high calorie or high fat meal. Acute administration can also be a single dose or multiple doses of a pharmaceutical composition administered around the onset of a craving for food, specifically a craving for fatty food. A time period close to or contemporaneous with the onset of an event will vary according to the event but can be, for example, within about 30 minutes of a meal or a craving for food. In certain embodiments, acute administration is administration within about an hour of a meal or a craving for food. In certain embodiments, acute administration is administration within about 2 hours, about 6 hours, about 10 hours, about 12 hours, about 15 hours or about 24 hours after a meal or a craving for food.

In a specific embodiment, the invention provides a method of preventing, treating, managing, or ameliorating a disorder, or one or more symptoms thereof, said methods comprising administering to a subject in need thereof compositions comprising an agonist or antagonist of a satiety factor once every 3 days, preferably, once every 4 days, once every 5 days, once every 6 days, once every 7 days, once every 8 days, once every 10 days, once every two weeks, once every three weeks, or once a month.

An effective amount of an agonist or antagonist of a satiety factor described herein will provide therapeutic benefit without causing substantial toxicity.

Toxicity of an agonist or antagonist of a satiety factor can be determined by standard pharmaceutical procedures in cell cultures or experimental animals, for example, by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1996, In: The Pharmacological Basis of Therapeutics, 9th ed., Chapter 2, p. 29, Elliot M. Ross).

5.4. Kits for Selecting Subjects with an Undesirable Level of a Satiety Factor

The invention also provides kits that are useful for selecting a subject for treatment with an agonist or antagonist of a satiety factor according to the present invention. In some embodiments, the kits of the present invention comprise a reagent that is capable of detecting a satiety factor or a plurality of satiety factors in a sample from a subject. The reagent may be an antibody or functional fragment or derivative thereof (e.g., Fab, F(ab)₂, Fv or sc Fv fragments) that specifically bind one or more satiety factors. In some embodiments, the antibodies may be detectably labeled. The reagent may be a part of an array, or the reagent may be packaged separately and/or individually. The kit may also comprise at least one internal standard to be used in determining the amount of one or more satiety factors.

In some embodiments, the kit can comprise one or more reagents capable of detecting one or more satiety factors and one or more agonists or antagonists of a satiety factor in an amount and form suitable for administration to a subject in need thereof. Useful reagents and useful agonists or antagonists are described in the sections above.

The kits of the present invention may also include reagents such as buffers that can be used in obtaining a sample from a subject. Prevention of the action of microorganism can be ensured by the inclusion of various antibacterial and antifungal agents, for example, paraben, chlorobutanol, phenol, sorbic acid and the like. It may also be desirable to include isotonic agents such as sugars, sodium chloride and the like.

In certain embodiments, the kits further comprise a label or labeling with instruction for carrying out a method of the invention. For example, the label of labeling can provide a normal satiety factor value. Further, the label or labeling can provide citations or links to sources of such reference amounts. In some embodiments, at least one positive control and at least negative control are included in the kit.

The following examples are offered to illustrate this invention and are not to be construed in any way as limiting the scope of this invention.

### 6. EXAMPLES

### 6.1. Example 1

### Treatment of Obesity with an Agonist or Antagonist of a Satiety Factor

6.1.1 Measuring the Amount of a Satiety Factor in a Blood Sample

Blood is collected from subjects into heparin-coated tubes containing 5000 kallikrein inhibitor units of aprotonin. Plasma is separated immediately by centrifugation at 4° C. and then stored at −70° C. until it is analyzed.

Antibodies against a satiety factor are obtained from available sources or generated utilizing standard techniques well known to those of skill in the art. Antibodies can be polyclonal or monoclonal.

The plasma level of the satiety factor is measured by immunoassay with radio-labeled antibodies or ELISA using standard technique known in the art.

6.1.2 Pharmaceutical Composition Comprising an Agonist or Antagonist of a Satiety Factor

A pharmaceutical composition comprising an effective amount of an agonist or antagonist of a satiety factor and one or more pharmaceutically acceptable carriers or excipients is prepared by standard methods known in the art. The pharmaceutical composition is in the form of a solution, suspension, emulsion, tablet, pill, capsule, powder, sustained-release formulation or the like.

6.1.3 Treating Obesity with an Agonist or Antagonist of a Satiety Factor

Pharmaceutical compositions comprising an agonist or antagonist of a satiety factor described above are used to treat obesity in a subject in need thereof. The subjects are selected based on the amount of a satiety factor in a sample from the subjects and a normal satiety factor value. In this example, the normal satiety factor value is established from a plurality of control subjects.

Determining Normal Satiety factor Value from Control Subjects

A physical examination, electrocardiogram, chemistry panel test, complete blood count or urinalysis is performed on men, aged 18-60 years, with a BMI from 20 kg/m² to 25 kg/m², according to the judgment of those of skill in the art. Based on these test and medical history, ten healthy men with normal BMI are chosen to determine their normal satiety factor values. Subjects who are taking regular medications are excluded from the study.

These subjects are fasted over night and are allowed water but no caloric beverages. On the next morning, a regular meal is presented to these subjects. One hour after the meal is presented, 5 ml samples of blood are collected. Each blood sample is prepared and processed as described above. The amount of satiety factor from each blood sample is determined using immunoassay.

The normal satiety factor value for fasted subjects is calculated as mean of the amount of satiety factor of samples taken from these subjects.

Selecting Subjects for Treating Obesity with an Agonist or Antagonist of a Satiety Factor

Ten obese (BMI from 30 kg/m² to 40 kg/m²), otherwise healthy men aged 18-60 years are screened for treatment with an agonist or antagonist of a satiety factor. The health condition of these subjects is determined by physical examination and chemistry panel test etc. as described above for control subjects.

These obese subjects are fasted and fed, and blood samples are taken as described above. The amount of satiety factor from each blood sample is determined and is compared to the normal satiety factor value determined above.

A subject is selected for treatment with an agonist or antagonist of the gut peptide if the amount of the gut peptide from his blood sample is less than 50% the normal gut peptide value determined above. Pharmaceutical compositions comprising an agonist or antagonist of a satiety factor are prepared as described above and administered intravenously or orally to the selected subject at the start of three meals per day for four weeks.

Electrocardiogram, chemistry panel test, complete blood count and urinalysis are performed to monitor the safety of the administration. Food intake, energy intake or expenditure, appetite, feeling of fullness, body weight, BMI, percentage of body fat and metabolic effects (such as sleeping metabolic rate, resting metabolic rate, fat oxidation and fat balance) etc. are monitored by practitioners of skill to determine the effectiveness and safety of the administration.

### 6.2. Example 2

### Treatment of Obesity with Enterostatin

6.2.1 Measuring the Amount of Enterostatin in a Blood Sample

The amount of enterostatin in a blood sample is measured using ELISA. ELISA using anti-APGPR antibodies can be performed as described in Imamura et al, 1998, Peptides, 19:8, 1385-1391; Bowyer et al., 1991, Clinica. Chimica. Acta. 200:137-152, the contents of which are incorporated hereby by reference in their entirety.

Collection of Blood Samples

5 ml blood sample is collected and immediately mixed with 20 mM zinc acetate and allowed to clot at room temperature for 30 minutes. The sample is then centrifuged at 3000 g for 20 minutes. The separated serum is mixed with an equal volume of ELISA immunoassay buffer containing 50 mM TRIS/HCL, 0.05% (w/v) casein, 3.1 mM NaN₃, 10 mM ethyenediaminetetraacetic acid (EDTA), and 0.05% (w/v) Tween 20 at pH 7.2-7.4. The sample is suspended in a boiling bath for 10 minutes and then centrifuged for 5 minutes at 10,000 g and the supernatant can be stored frozen at −70° C. until assayed for enterostatin.

The stored aliquots of serum can be later thawed at room temperature, thoroughly mixed and centrifuged for five minutes at 10,000 g. To extract enterostatin, the supernatant is mixed with 1:9 volume of methanol. The mixture can be stored over ice for 30-60 minutes and then centrifuged at 11,000 g for 10 minutes at 4° C. The clear supernatant can be lyophilized and suspended in ELISA buffer for assay or in TBS (50 mM Tris.HCl, 150 nM NaCl, 3.1 mM NaN₃, pH 7.4) for chromatography. To inhibit proteolytic degradation of enterostatins during the assay, two protease inhibitors can be added to the serum samples before ELISA (final concentration, 1 mmol/L diprotein A and 0.1 mmol/L captopril).

Enzyme-Linked Immunosorbent Assay (ELISA)

Antibodies against APGPR can be generated utilizing standard techniques well know to those skill in the art. Antibodies can be polyclonal or monoclonal.

Preparation of Coating Antigen: 1 ml of a 5 mg bis(sulfosuccinimidyl) suberate (BS) (Pierce, Ill., USA) in PBS, pH 7.2 is slowly added dropwise to 2 ml of 10 g/l rabbit serum albumin (RSA) in PGS and stirred for 2 hours at room temperature. Excess BS is removed by gel filtration in PBS on a 2-20 cm Sephadex G-50 Column. The protein peak, as monitored by absorbance at 280 nm, is pooled. This is incubated overnight at 4° C. with two changes of dialysis buffer. The protein content is measured using a Lowry method (See e.g., Markwell et al., 1978, Anal. Biochem. 87:206-210) as 730 μg/ml This is diluted to 0.5 mg/ml and 3.1 mmol/l NaN₃ is added. Portions are stored at −20° C. till required.

Competitive ELISA: the wells of PVC microtitre plates are coated with 100 μl of 0.2 μg/ml RSA-BS-CCG-APGPR and 0.8 μg/ml RSA in 15 mmol/l Na₂CO₃, 35 mmol/l NaHCO₃, 3.1 mmol/l NaN₃ pH 9.6 by incubation overnight at 4° C. All further incubation are done at room temperature on an agitator. The plates are then washed three times and blocked with wash buffer (20 mmol/l Tris/HCl, 75 mmol/l NaCl, 3.1 mmol/l NaN₃, 0.05% (w/v) Tween 20 at pH 7.2-7.4). Then 100 μl of either unknown or standard synthetic APGPR peptide (purchased from American Peptide Company) solutions plus 50 μl 1:2000 mouse anti-APGPR monoclonal antibody in ELISA buffer (50 mM TRIS/HCL, 0.05% (w/v) casein, 3.1 mM NaN₃, 10 mM ethyenediaminetetra-acetic acid (EDTA), and 0.05% (w/v) Tween 20 at pH 7.2-7.4) are incubated in the wells for one hour. Between each incubation the plates are washed three times in wash buffer. Firstly 100 μl of 1:1000 goat anti-mouse IgG biotin conjugate in ELISA buffer is incubated in each well for 30 minutes, then 100 μl of 1:500 extravidin alkaline phosphatase solution in wash buffer is incubated in each well for 30 minutes. Finally 100 μl of 1 mg/ml p-nitrophenyl phosphate in substrate buffer (10% (w/v) diethanolamine/HCl, 0.49 mmol/l MgCl₂, 3.1 mmol/l NaN₃, pH 9.8) is incubated in each well until the maximum absorbance at 405 nm measured on an Anthos 2001 ELISA plate reader is 1.5 for the minimum standard peptide concentration. Reaction is terminated by adding 3 mmol/l NaOH (50 μl). The plate then read at 405 nm and a standard curve is constructed to calibrate the readings.

A standard inhibition curve under the specified conditions is obtained by plotting the concentration of competing synthetic antigen (APGPR) on the x axis, which is a log scale, against absorbance on the y axis, which is a linear scale. The concentration of antigen (APGPR) in a sample from a subject can be interpolated from the standard antigen-inhibition curve.

Chromatography Analysis

To ascertain the size of serum APGPR immunoreactivity, gel filtration chromatography is performed using Sephadex G-25 (50×1.0 m; 9.3 ml; fractionation range for globular proteins, 1-5 kDa) column. Lyophilized methanol-extracted serum reconstituted in a minimal volume of distilled water is loaded on columns equilibrated with buffer A (10 mmol/l NH₄HCO₃). The column is eluted with 10 mmol/l NH₄HCO₃ at a rate of about 5 minute/1 ml fraction.

6.2.2 Oral Dosage Forms of Enterostatin

Oral dosage forms comprising enterostatin or co-complexes comprising enterostatin are prepared.

Enterostatin is produced under Good Manufacture Procedures (cGMP) by American Peptide Company. The purity of enterostatin is greater than 95% by HPLC analysis.

Enterostatin co-complexes can be prepared as described in U.S. provisional application No. 60/750,208, the content of which is incorporated by reference in its entirety. For example, enterostatin co-complexes by combining a co-crystal guest and an enterostatin in a 1:1 molar ratio in a solvent. The solvents are allowed to evaporate and the resulting solid co-complexes is collected. The solvent can be methanol, the salt is enterostatin acetate and the guest is 1-hydroxy-2-naphthoic acid. The resulting solid is in the form of light brown flakes or broken glass.

Oral dosage forms of enterostatin may contain 2.5, 4, 5, 7.5, 10, 15, 20, 30, 40, 50, 60, or 70 mg enterostatin. They may comprise excipients or non-hygroscopic additives. Suitable excipients may be starches, sugars and micro-crystalline cellulose etc. Suitable non-hygroscopic may be dibasic calcium phosphate anhydrous, calcium sulfate, powdered cellulose, dextrose and lactitol etc. Oral dosage forms of enterostatin may be in the form of tablets or capsules.

Exemplary capsules comprising enterostatin may contain a fill with 2.5% enterostatin (% weight), 42% Cremphor EL, 20% Labrasol, and 30% labrafil M2125CS, and a shell with 54% Gelatin, 18% Glycerin, 22% anidrisorb 35/70, and 6% water.

6.2.3 Treating Obesity with Enterostatin

Oral dosage forms of enterostatin described above are used to treat obesity in a subject in need thereof. The subjects are selected based on the amount of enterostatin in a sample from the subjects and a normal enterostatin value. In this example, the normal enterostatin value is established from a plurality of control subjects.

Selecting a Patient for Treating Obesity with Enterostatin

Determining Normal Enterostatin Value from Control Subjects

A physical examination, electrocardiogram, chemistry panel test, complete blood count and urinalysis are performed on men, aged 18-60 years, with a BMI from 20 kg/m² to 25 kg/m², according to the judgment of those of skill in the art. Based on these test and medical history, ten healthy men with normal BMI are chosen to determine the normal enterostatin value. Subjects who are taking regular medications are excluded from the study.

These subjects are fasted over night and are allowed water but no caloric beverages. On the next morning, a high fat meal is presented to these subjects. The high fat meal contains about 800 cal and contains 45% fat. Three hours after the meal is presented, 5 ml samples of blood are collected via an indwelling catheter from the subjects. Each blood sample is prepared and processed as described above. The amount of enterostatin from each blood sample is determined described above.

The normal enterostatin value for fasted subjects is calculated as mean of the amount of enterostatin of samples taken from these subjects.

Selecting Subjects for Treating Obesity with Enterostatin

Ten obese (BMI from 30 kg/m² to 40 kg/m²), otherwise healthy men aged 18-60 years are screened for treatment with enterostatin. The health condition of these subjects are determined by physical examination and chemistry panel test etc. as described above for control subjects.

These obese subjects are fasted and fed, and blood samples are taken as described above. The amount of enterostatin from each blood sample is determined using ELISA described above and is compared to the normal enterostatin value determined above.

A subject is selected for treatment with enterostatin if the amount of enterostatin from his blood sample is less than half of the normal enterostatin value determined above. Capsules for oral administration comprising 20.0 mg enterostatin are prepared as described above. The enterostatin capsules are given to the selected subject at the start of three meals per day for four weeks.

Electrocardiogram, chemistry panel test, complete blood count and urinalysis are performed to monitor the safety of oral enterostatin administration. Food intake, energy intake or expenditure, appetite, feeling of fullness, body weight, BMI, percentage of body fat and metabolic effects (such as sleeping metabolic rate, resting metabolic rate, fat oxidation and fat balance) etc. are monitored by practitioners of skill to determine the effectiveness and safety of oral enterostatin administration.

### 6.3. Example 3

### Treatment of Obesity with Peptide YY₃₋₃₆ (PYY₃₋₃₆)

6.3.1 Measuring the Amount of Peptide YY₃₋₃₆ in a Blood Sample

The plasma level of PYY₃₋₃₆ is measured by immunoassay with radio-labeled antibodies. Immunoassay using radio-labeled anti-PYY antibodies can be performed as described in Batterham et al., 2003, New Eng. J. Med. 349(10):941-8, the contents of which are incorporated by reference in their entirety.

Blood is collected from subjects into heparin-coated tubes containing 5000 kallikrein inhibitor units of aprotonin. Plasma is separated immediately by centrifugation at 4° C. and then stored at −70° C. until it is analyzed.

Antibodies against PYY can be generated utilizing standard techniques well know to those skill in the art. Antibodies can be polyclonal or monoclonal. The antibodies against PYY are produced in a rabbit against synthetic porcine PYY coupled to bovine serum albumin by glutaraldehyde and used at a final dilution of 1:50,000. The antibodies can detect both PYY and PYY₃₋₃₆. I¹²⁵-labeled PYY antibodies are prepared by the iodogen method and purified by high-pressure liquid chromatography. All blood samples are assayed in duplicate and 200 μl of unextracted plasma is assayed. The assay is performed in a 700 μl of 0.06 M phosphate buffer (pH 7.3), containing 0.3 percent bovine serum albumin. 200 μl of PYY-free plasma is used as control. The sample is incubated for three days at 4° C. before the separation of free and antibody-bound label by sheep antirabbit antibody. The assay can detect changes of 2 pmol/l PYY in samples.

6.3.2 Pharmaceutical Composition Comprising PYY₃₋₃₆

PYY₃₋₃₆ is produced under Good Manufacture Procedures (cGMP) by Phoenix Pharmaceuticals Inc. The purity of PYY₃₋₃₆ is greater than 95% by HPLC analysis. Pharmaceutical compositions comprising PYY₃₋₃₆ can be prepared and formulated as described in U.S. Pat. App. No. 20050009748.

6.3.3 Treating Obesity with PYY₃₋₃₆

Pharmaceutical compositions comprising PYY₃₋₃₆ described above are used to treat obesity in a subject in need thereof. The subjects are selected based on the amount of PYY in a sample from the subjects and a normal PYY value. In this example, the normal PYY value is established from a plurality of control subjects.

Determining Normal PYY Value from Control Subjects

A physical examination, electrocardiogram, chemistry panel test, complete blood count and urinalysis are performed on men, aged 18-60 years, with a BMI from 20 kg/m² to 25 kg/m², according to the judgment of those of skill in the art. Based on these test and medical history, ten healthy men with normal BMI are chosen to determine the normal PYY value. Subjects who are taking regular medications are excluded from the study.

These subjects are fasted over night and are allowed water but no caloric beverages. On the next morning, a regular meal is presented to these subjects. One hour after the meal is presented, 5 ml samples of blood are collected. Each blood sample is prepared and processed as described above. The amount of PYY from each blood sample is determined according to above.

The normal PYY value for fasted subjects is calculated as mean of the amount of PYY of samples taken from these subjects.

Selecting Subjects for Treating Obesity with PYY

Ten obese (BMI from 30 kg/m² to 40 kg/m²), otherwise healthy men aged 18-60 years are screened for treatment with PYY₃₋₃₆. The health condition of these subjects is determined by physical examination and chemistry panel test etc. as described above for control subjects.

These obese subjects are fasted and fed, and blood samples are taken as described above. The amount of PYY from each blood sample is determined using methods described above and is compared to the normal PYY value determined above.

A subject is selected for treatment with PYY₃₋₃₆ if the amount of PYY₃₋₃₆ from his blood sample is less than 70% the normal PYY₃₋₃₆ value determined above. Pharmaceutical compositions comprising PYY₃₋₃₆ are prepared as described above and are administered orally to the selected subject at the start of three meals per day for four weeks. Electrocardiogram, chemistry panel test, complete blood count and urinalysis are performed to monitor the safety of oral PYY₃₋₃₆ administration. Food intake, energy intake or expenditure, appetite, feeling of fullness, body weight, BMI, percentage of body fat and metabolic effects (such as sleeping metabolic rate, resting metabolic rate, fat oxidation and fat balance) etc. are monitored by practitioners of skill to determine the effectiveness and safety of oral PYY₃₋₃₆ administration.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What claimed is:

1. A method of treating or preventing a disorder or condition associated with an undesirable level of a satiety factor in a subject, comprising administering to the subject an amount of an agonist or antagonist of said satiety factor effective for treating or preventing the disorder or condition wherein said subject has said undesirable level of the satiety factor.

2. The method of claim 1, wherein the disorder or condition is selected from overweight, obesity, metabolic disorders, hypertension, lipid related disorders, anorexia and type II diabetes.

3. The method of claim 2, wherein the disorder or condition is overweight.

4. The method of claim 2, wherein the disorder or condition is obesity.

5. The method of claim 2, wherein the disorder or condition is type II diabetes.

6. The method of claim 1, wherein the subject is a human.

7. The method of claim 6, wherein the subject has a Body Mass Index (“BMI”) greater than 25 kg/m².

8. The method of claim 6, wherein the subject has a Body Mass Index (“BMI”) greater than 30 kg/m².

9. The method of claim 6, wherein the subject has a Body Mass Index (“BMI”) greater than 35 kg/m².

10. The method of claim 6, wherein the subject has a Body Mass Index (“BMI”) less than 25 kg/m².

11. The method of claim 6, wherein the subject has a Body Mass Index (“BMI”) less than 22 kg/m².

12. The method of claim 6, wherein the subject has a Body Mass Index (“BMI”) less than 20 kg/m².

13. The method of claim 1, wherein said satiety factor is a peptide.

14. The method of claim 1, wherein said satiety facotr is selected from the group consisting of adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β-endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), melanocyte stimulating hormone (α-MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptide, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

15. The method of claim 14, wherein said proglucagon-derived peptide is glucagon, glucagon-like peptide 1 (GLP-1), glucagon-like peptide 2 (GLP-2), oxyntomodulin, glicentin, glicentin-related pancreatic peptide or major proglucagon fragment.

16. The method of claim 1, wherein said satiety factor is non-gut peptide.

17. The method of claim 1, wherein said satiety factor is a gut peptide.

18. The method of claim 17, wherein said satiety factor is selected from the group consisting of: amylin, bombesin or bombesin-like peptide, cholecystokinin, enterostatin, ghrelin, glucagon-like peptide 1, obestatin, oxyntomodulin, pancreatic polypeptide and peptide YY.

19. The method of claim 1, wherein the subject has an undesirable level of said satiety factor when the amount of said satiety factor in a sample from the subject is below a normal value.

20. The method of claim 19, wherein said satiety factor is selected from the group consisting of: amylin, bombesin or bombesin-like peptide, cholecystokinin, enterostatin, glucagon-like peptide 1, obestatin, oxyntomodulin, pancreatic polypeptide and peptide YY.

21. The method of claim 19, wherein an agonist of said satiety factor in an amount effective for treating or preventing the disorder or condition is administered to the subject.

22. The method of claim 1, wherein the subject has an undesirable level of said satiety factor when the amount of said satiety factor in a sample from the subject is above a normal value.

23. The method of claim 22, wherein an antagonist of said satiety factor in an amount effective for treating or preventing the disorder or condition is administered to the subject.

24. The method of claim 1, wherein the administration is oral, intranasal, intrapulmonary, intravenous, subcutaneous, transdermal, intragastric, intraperitoneal, intracerebroventricular or rectal.

25. The method of claim 1, wherein the agonist or antagonist of said satiety factor is administered prior to a meal.

26. The method of claim 1, wherein the agonist or antagonist of said satiety factor is administered around a meal time.

27. The method of claim 1, wherein the agonist or antagonist of said satiety factor is administered continuously.

28. The method of claim 1, wherein an agonist or antagonist of a gut peptide satiety factor is administered with an agonist or antagonist of a non-gut peptide satiety factor.

29. A method of selecting a subject for treatment with an agonist or antagonist of a satiety factor, comprising the step of determining the amount of said satiety factor in a sample from the subject, wherein the subject is selected for treatment when the amount of said satiety factor in the sample of the subject is above or below a normal value.

30. The method of claim 29, wherein the subject is a human.

31. The method of claim 29, wherein the sample is selected from a blood sample, a plasma sample, a saliva sample, a serum sample, a sputum sample, a urine sample, a cell sample, a cellular extract sample and a tissue biopsy sample.

32. The method of claim 29, wherein the amount of said satiety factor in the sample from the subject is determined by spectrometry, chromatography, immunoassay or electrophoresis.

33. The method of claim 29, wherein the amount of said satiety factor is determined when the subject is fasted.

34. The method of claim 29, wherein the amount of said satiety factor determined when the subject is fed.

35. The method of claim 29, wherein said satiety factor is a peptide.

36. The method of claim 29, wherein said satiety facotr is selected from the group consisting of adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), a melanocyte stimulating hormone (MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptide, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

37. The method of claim 36, wherein said proglucagon-derived peptide is glucagon, glucagon-like peptide 1 (GLP-1), glucagon-like peptide 2 (GLP-2), oxyntomodulin, glicentin, glicentin-related pancreatic peptide or major proglucagon fragment.

38. The method of claim 29, wherein said satiety factor is a non-gut peptide.

39. The method of claim 29, wherein said satiety factor is a gut peptide.

40. The method of claim 39, wherein said satiety factor is selected from the group consisting of: amylin, bombesin or bombesin-like peptide, cholecystokinin, enterostatin, ghrelin, glucagon-like peptide 1, obestatin, oxyntomodulin, pancreatic polypeptide and peptide YY.

41. The method of claim 29, wherein the subject is selected for treatment when the amount of said satiety factor in a sample from the subject is below a normal value.

42. The method of claim 41, wherein said satiety factor is selected from the group consisting of: amylin, bombesin or bombesin-like peptide, cholecystokinin, enterostatin, glucagon-like peptide 1, obestatin, oxyntomodulin, pancreatic polypeptide and peptide YY.

43. The method of claim 29, wherein the subject is selected for treatment when the amount of said satiety factor in a sample from the subject is above a normal value.

44. A method of treating or preventing a disorder or condition associated with an undesirable level of a satiety factor in a subject, comprising (a) selecting a subject with an undesirable level of a satiety factor for treatment; and (b) administering to the subject an amount of an agonist or antagonist of said satiety factor effective for treating or preventing the disorder or condition.

45. The method of claim 44, wherein the disorder or condition is selected from overweight, obesity, metabolic disorders, hypertension, lipid related disorders, anorexia and type II diabetes.

46. The method of claim 45, wherein the disorder or condition is overweight.

47. The method of claim 45, wherein the disorder or condition is obesity.

48. The method of claim 45, wherein the disorder or condition is type II diabetes.

49. The method of claim 44, wherein the subject is a human.

50. The method of claim 49, wherein the subject has a Body Mass Index greater than 25 kg/m².

51. The method of claim 49, wherein the subject has a Body Mass Index greater than 30 kg/m².

52. The method of claim 49, wherein the subject has a Body Mass Index greater than 35 kg/m².

53. The method of claim 49, wherein the subject has a Body Mass Index less than 25 kg/m².

54. The method of claim 49, wherein the subject has a Body Mass Index less than 22 kg/m².

55. The method of claim 49, wherein the subject has a Body Mass Index less than 20 kg/m².

56. The method of claim 49, wherein said satiety factor is a peptide.

57. The method of claim 56, wherein said satiety facotr is selected from the group consisting of adiponectin, agouti-related protein (AGRP), amylin, apolipoprotein A-IV, beacon, bombesin or bombesin like peptide, brain derived neural factor (BDNF), calcitonin-gene related peptide (CGRP), β casomorphin, cholecystokinin (CCK), ciliary neurotrophic factor (CNTF), cocaine and amphetamine regulated transcript (CART), corticotropin-releasing hormone (CRH), cyclo-his-pro, dynorphin, β-endorphin, enterostatin, galanin, galanin-like peptide (GALP), ghrelin, growth hormone-releasing hormone (GHRH), hypocretins/orexins, insulin, insulin like growth factor I and II (IGF-I and IGF-II), leptin, melanin concentrating hormone (MCH), melanocyte stimulating hormone (α-MSH), motilin, nesfatin, neuromedin B and neuromedin U, neuropeptide B (NPB) and (NPW), neuropeptide K (NPK), neuropeptide Y (NPY), neurotensin (NT), obestatin, oxytocin, pancreatic peptide, peptide YY, proglucagon-derived peptide, prolactin-releasing peptide, pro-opiomelanocortin (POMC), protoporphyrin, QRFP 43 (an RF amide peptide, 26Rfa), somatostatin, thyrotropin-releasing hormone (TRH), urocortin, and vasopressin.

58. The method of claim 57, wherein said proglucagon-derived peptide is glucagon, glucagon-like peptide 1 (GLP-1), glucagon-like peptide 2 (GLP-2), oxyntomodulin, glicentin, glicentin-related pancreatic peptide or major proglucagon fragment.

59. The method of claim 56, wherein said satiety factor is a non-gut peptide.

60. The method of claim 56, wherein said satiety factor is a gut peptide.

61. The method of claim 60, wherein said satiety factor is selected from the group consisting of: amylin, bombesin or bombesin-like peptide, cholecystokinin, enterostatin, ghrelin, glucagon-like peptide 1, obestatin, oxyntomodulin, pancreatic polypeptide and peptide YY.

62. The method of claim 44, wherein the method further comprises determining the amount of said satiety factor in a sample from the subject.

63. The method of claim 44, wherein the sample is selected from a blood sample, a plasma sample, a saliva sample, a serum sample, a sputum sample, a urine sample, a cell sample, a cellular extract sample and a tissue biopsy sample.

64. The method of claim 44, wherein the amount of said satiety factor in the sample from the subject is determined by spectrometry, chromatography, immunoassay or electrophoresis.

65. The method of claim 44, wherein the amount of said satiety factor is determined when the subject is fasted.

66. The method of claim 44, wherein the amount of said satiety factor is determined when the subject is fed.

67. The method of claim 44, wherein the subject is selected for treatment when the amount of said satiety factor in a sample from the subject is below a normal value.

68. The method of claim 67, wherein said satiety factor is selected from the group consisting of: amylin, bombesin or bombesin-like peptide, cholecystokinin, enterostatin, ghrelin, glucagon-like peptide 1, obestatin, oxyntomodulin, pancreatic polypeptide and peptide YY.

69. The method of claim 67, wherein an agonist of said satiety factor in an amount effective for treating or preventing the disorder or condition is administered.

70. The method of claim 44, wherein the subject is selected for treatment when the amount of said satiety factor in a sample from the subject is above a normal value.

71. The method of claim 44, wherein an agonist or antagonist of a gut peptidesatiety factor is administered with an agonist or antagonist of a non-gut peptide satiety factor.

72. A method of reducing food intake in a subject, comprising (a) selecting a subject with an undesirable level of a satiety factor; and (b) administering to the subject an amount of an agonist or antagonist of said satiety factor effective for reducing food intake.

73. The method of claim 72 wherein said food comprises fat.

74. The method of claim 72 wherein said food is fat.

75. The method of claim 72 wherein said food comprises carbohydrate.

76. The method of claim 72 wherein said food is carbohydrate.

77. The method of claim 72 wherein said food comprises protein.

78. The method of claim 72 wherein said food is protein.

79. A method of selecting a subject for treatment with one or more agonists or antagonists of satiety factors, comprising the step of determining the amounts of satiety factors in a sample from the subject, wherein the subject is selected for treatment when the amounts of one or more satiety factors in the sample of the subject are independently above or below normal values.

80. The method of claim 79 wherein the amounts of a panel of satiety factors in a sample from the subject are determined.

81. The method of claim 80 wherein the panel comprises two, three, four, five, six, seven, eight, nine or ten satiety factors selected from the group consisting of: amylin, bombesin or bombesin-like peptide, cholecystokinin, enterostatin, ghrelin, glucagon-like peptide 1, obestatin, oxyntomodulin, pancreatic polypeptide and peptide YY.

82. The method of claim 79 further comprising the step of administering to the subject one or more agonists or antagonists of the satiety factors having amounts above or below normal values.

83. The method of claim 82 wherein more than one agonist or antagonist is administered to the subject.

84. The method of claim 82 wherein an agonist or antagonist of a gut peptide is administered with an agonist or antagonist of a non-gut peptide

85. A method of treating obesity in a subject, comprising (a) selecting a subject an undesirable level of a satiety factor; and (b) administering to the subject an amount of an agonist or antagonist of said satiety factor effective for treating obesity.

86. A kit for selecting a subject for treatment of obesity with an agonist or antagonist of a satiety factor, comprising a device capable of obtaining a fluid of the subject and a reagent capable of detecting said satiety factor in the fluid.

87. A kit for treating or preventing obesity in a subject, comprising a reagent capable of detecting a satiety factor in a fluid of the subject and an effective amount of an agonist or antagonist of the satiety factor.
